SEQUENCE LISTING (19 sequences)

SEQ ID NO: 1 - protein, 20 aa, Artificial Sequence
  source: /note=“Description of Artificial Sequence Synthetic peptide”
MARRPRHSIYSSDEDDEDFE

SEQ ID NO: 2 - protein, 19 aa, Artificial Sequence
  source: /note=“Description of Artificial Sequence Synthetic peptide”
GGGSGGSWGEGCDRSGKYG

SEQ ID NO: 3 - protein, 25 aa, Artificial Sequence
  source: /note=“Description of Artificial Sequence Synthetic peptide”
QMGIVSWGEGCDRDGKYGFYTHVFR

SEQ ID NO: 4 - protein, 17 aa, Artificial Sequence
  source: /note=“Description of Artificial Sequence Synthetic peptide”
AISGSGGSTYYADSVKG

SEQ ID NO: 5 - protein, 17 aa, Artificial Sequence
  source: /note=“Description of Artificial Sequence Synthetic peptide”
AASGSGGSTYYADSVKG

SEQ ID NO: 6 - protein, 17 aa, Artificial Sequence
  source: /note=“Description of Artificial Sequence Synthetic peptide”
AIAGSGGSTYYADSVKG

SEQ ID NO: 7 - protein, 17 aa, Artificial Sequence
  source: /note=“Description of Artificial Sequence Synthetic peptide”
AISAAGGSTYYADSVKG

SEQ ID NO: 8 - protein, 17 aa, Artificial Sequence
  source: /note=“Description of Artificial Sequence Synthetic peptide”
AISGASGSTYYADSVKG

SEQ ID NO: 9 - protein, 17 aa, Artificial Sequence
  source: /note=“Description of Artificial Sequence Synthetic peptide”
AISGSAGSTYYADSVKG

SEQ ID NO: 10 - protein, 17 aa, Artificial Sequence
  source: /note=“Description of Artificial Sequence Synthetic peptide”
AISGSGASTYYADSVKG

SEQ ID NO: 11 - protein, 17 aa, Artificial Sequence
  source: /note=“Description of Artificial Sequence Synthetic peptide”
AISGSGGATYYADSVKG

SEQ ID NO: 12 - protein, 17 aa, Artificial Sequence
  source: /note=“Description of Artificial Sequence Synthetic peptide”
AISGSGGSAYYADSVKG

SEQ ID NO: 13 - protein, 17 aa, Artificial Sequence
  source: /note=“Description of Artificial Sequence Synthetic peptide”
AISGSGGSTAYADSVKG

SEQ ID NO: 14 - protein, 17 aa, Artificial Sequence
  source: /note=“Description of Artificial Sequence Synthetic peptide”
AISGSGGSTYAADSVKG

SEQ ID NO: 15 - protein, 17 aa, Artificial Sequence
  source: /note=“Description of Artificial Sequence Synthetic peptide”
AISGSGGSTYYAASVKG

SEQ ID NO: 16 - protein, 17 aa, Artificial Sequence
  source: /note=“Description of Artificial Sequence Synthetic peptide”
AISGSGGSTYYADAVKG

SEQ ID NO: 17 - protein, 17 aa, Artificial Sequence
  source: /note=“Description of Artificial Sequence Synthetic peptide”
AISGSGGSTYYADSAKG

SEQ ID NO: 18 - protein, 17 aa, Artificial Sequence
  source: /note=“Description of Artificial Sequence Synthetic peptide”
AISGSGGSTYYADSVAG

SEQ ID NO: 19 - protein, 17 aa, Artificial Sequence
  source: /note=“Description of Artificial Sequence Synthetic peptide”
AISGSGGSTYYADSVKA

(19) United States
(12) Patent Application Publication
(10) Pub. No.: US 2021/0032368 A1
(43) Pub. Date: Feb. 4, 2021

(54) METHODS AND COMPOSITIONS FOR THE DEVELOPMENT OF ANTIBODIES SPECIFIC TO EPITOPE POST-TRANSLATIONAL MODIFICATION STATUS

(71) Applicant: Abcam plc, Cambridge (GB)

(72) Inventors: Michael Weiner, Cambridge (GB); Margaret Kiss, Cambridge (GB)

(21) Appl. No.: 16/635,123

(22) PCT Filed: Aug. 3, 2018
(86) PCT No.: PCT/IB18/01005

§ 371 Date: Jan. 29, 2020

Related U.S. Application Data
(60) Provisional application No. 62/541,530, filed on Aug. 4, 2017.

(51) Int. Cl. C07K 16/44; C07K 16/32

(52) U.S. Cl. CPC C07K 16/44; C07K 2317/622; C07K 2317/10; C07K 16/32

(57) ABSTRACT

The present disclosure provides, among other things, a method of generating antibodies that recognize a protein of interest. In some aspects, the protein of interest contains a post translational modification (PTM) site. Provided in some aspects is a method of generating non-PTM-binding antibodies that specifically bind a site without post translational modification. Provided in some aspects is a pan-PTM-binding antibody library comprising a plurality of antibodies derived from a pre-existing antibody that specifically recognizes a PTM on a peptide or protein of interest. Provided in further aspects is a non-PTM-binding antibody library comprising a plurality of antibodies derived from a pre-existing antibody that specifically recognizes a PTM on a peptide or protein of interest.

### RELATED APPLICATIONS

This application claims priority to U.S. Provisional Application Ser. No. 62/541,530, filed on Aug. 4, 2017, the disclosure of which is hereby incorporated by reference in its entirety.

### BACKGROUND

Many cellular activities are controlled by post-translational modifications, such as phosphorylation. However, studies of the role of these modifications in cell growth, function, development, and differentiation are hampered by the lack of specific reagents. The most commonly used method for generating antibodies (“Abs”) is through immunization of animals. However, this method is generally low-throughput, expensive, time-consuming, and the Abs generated are not always renewable. The rarity of post-translational modification specific Ab clones adds to the challenge.

Traditional methods for generating antibodies against post-translational modification antigens are currently not efficient. Moreover, there are no current methods to develop Ab pairs where one member of the pair binds to the modified epitope and the second member of the pair binds to the same sequence, unmodified.

Thus, there remains a need for improved methods and compositions for the development of antibodies that recognize, bind to, and/or modulate epitopes that have specific post-translational modification status.

### SUMMARY OF THE INVENTION

Provided herein are methods that allow for the development of antibodies that recognize, bind to, and or modulate epitopes having specific post-translational modifications. The current disclosure for the first time describes a structure-based directed evolution approach to the production of post-translational modification specific antibodies. In some embodiments described herein are methods to produce focused antibody libraries based on an antibody framework with a pre-existing propensity to bind specific post-translationally modified epitopes and a general mode to bind context sequences.

The present disclosure provides, among other things, a method of generating antibodies that recognize a post translational modification (PTM) site independent of PTM status comprising: providing an antibody that specifically recognizes a PTM on a peptide or protein of interest; identifying a PTM binding pocket of the antibody; generating a library comprising candidate pan-PTM binding antibodies by randomizing one or more regions inside or outside the PTM binding pocket that bind to a context sequence adjacent to the PTM site; screening the library against the peptide or protein of interest without PTM, thereby identifying pan-PTM binding antibodies.

The present disclosure provides, among other things, a method of generating antibodies that recognize a post translational modification (PTM) site, either regardless of modification status or dependent on modification status, comprising: providing an antibody that specifically recognizes a PTM on a peptide or protein of interest; identifying a PTM binding pocket of the antibody; introducing a non-charged amino acid at one or more sites within the PTM binding pocket of the antibody that are determined to interact with the PTM; generating a library comprising candidate pan-PTM binding antibodies by randomizing one or more regions inside or outside the PTM binding pocket that bind to a context sequence adjacent to the PTM site; screening the library against the peptide or protein of interest without PTM, thereby identifying pan-PTM binding antibodies.

In some embodiments, the PTM site is a naturally occurring PTM site. In some embodiments, the PTM site is an engineered PTM site. In some embodiments, the engineered PTM site is introduced into a peptide or protein of interest by site-specific mutagenesis. Various manners known in the art can be used to produce the engineered PTM site. In some embodiments, the site-specific mutagenesis comprises a point mutation, a series of point mutations, a deletion or an insertion. In some embodiments, the engineered PTM site is introduced by one or more amino acid substitutions in the peptide or protein of interest. In some embodiments, the engineered PTM site is introduced by insertion of one or more amino acids into the peptide or protein of interest. In some embodiments, the one or more amino acids inserted into the peptide or protein of interest comprises about 1, 2, 3, 4, 5, 6, 7, 8, 9, 10, 11, 12, 13, 14, 15, 16, 17, 18, 19, 20, 21, 22, 23, 24 or 25 amino acids. In some embodiments, the one or more amino acid substitutions can be 1, 2, 3, 4, 5, 6, 7, 8, 9, 10, 11, 12, 13, 14, 15, 16, 17, 18, 19, 20, 21, 22, 23, 24 or 25 amino acid substitutions.

In some embodiments, the generated antibodies recognize a PTM site regardless of modification status. In some embodiments, the generated antibodies recognize a PTM site dependent on the modification status. For example, in some embodiments, the modification status can be lack of PTM.

In some embodiments, the one or more sites within the PTM binding pocket are structurally-predicted. In some embodiments, the one or more sites within the PTM binding pocket are experimentally-determined. In some embodiments, the non-charged amino acid is alanine or glycine, although any non-charged amino acid can be used. Various manners in the art can be used to introduce the non-charged amino acid. Any suitable method to introduce the non-charged amino acid can be used. In some embodiments, non-charged amino acid is introduced by site mutagenesis.

The methods herein can be applied to any PTM. For example, the PTM can be any kind of acetylation, amidation, deamidation, prenylation (such as farnesylation or geranylation), formylation, glycosylation, hydroxylation, methylation, myristoylation, nitrosylation, phosphorylation, sialylation, sulphation, polysialylation, ubiquitination, SUMOylation, NEDDylation, ribosylation, sulphation, or any combinations thereof. In some embodiments, the PTM is negatively charged, positively charged, hydrophilic and/or hydrophobic. In some embodiments, the PTM is phosphorylation. In some embodiments, the PTM is glycosylation. In some embodiments, the glycosylation is sialylation, acetylation or methylation.

In one aspect, the present disclosure provides, among other things, a method of generating non-PTM-binding antibodies that specifically bind a PTM site in the absence of post translational modification comprising; providing an antibody that specifically recognizes a PTM on a peptide or protein of interest; identifying a PTM binding pocket of the antibody; generating a library comprising candidate non-PTM-binding antibodies by randomizing one or more regions inside or outside the PTM binding pocket that bind to a context sequence adjacent to the PTM site; screening the library against the peptide or protein of interest without PTM, thereby identifying non-PTM binding antibodies.

In one aspect, the present disclosure provides, among other things, a method of generating non-PTM-binding antibodies that specifically binds to a site that has the possibility of becoming post-translationally modified, but only when such site has not been modified by a post translational modification (PTM) comprising: providing an antibody that specifically recognizes a PTM on a peptide or protein of interest; identifying a PTM binding pocket of the antibody; introducing an amino acid that repels the PTM at one or more sites in the PTM binding pocket of the antibody that are determined to interact with the PTM; generating a library comprising candidate non-PTM binding antibodies by randomizing one or more regions either inside or outside the PTM binding pocket that bind to a context sequence adjacent to the PTM site; and screening the library against the peptide or protein of interest without PTM, thereby identifying non-PTM binding antibodies.

In some embodiments, the PTM site is a naturally occurring PTM site. In some embodiments, the PTM site is an engineered PTM site. In some embodiments, the engineered PTM site is introduced into a peptide or protein of interest by site-specific mutagenesis. Various manners known in the art can be used to produce the engineered PTM site. In some embodiments, the site-specific mutagenesis comprises a point mutation, a series of point mutations, a deletion or an insertion. In some embodiments, the engineered PTM site is introduced by one or more amino acid substitutions in the peptide or protein of interest. In some embodiments, the engineered PTM site is introduced by insertion of one or more amino acids into the peptide or protein of interest. In some embodiments, the one or more amino acids inserted into the peptide or protein of interest comprises about 1, 2, 3, 4, 5, 6, 7, 8, 9, 10, 11, 12, 13, 14, 15, 16, 17, 18, 19, 20, 21, 22, 23, 24 or 25 amino acids. In some embodiments, the one or more amino acid substitutions can be 1, 2, 3, 4, 5, 6, 7, 8, 9, 10, 11, 12, 13, 14, 15, 16, 17, 18, 19, 20, 21, 22, 23, 24 or 25 amino acid substitutions.

In some embodiments, one or more sites within the PTM binding pocket are structurally-predicted. In some embodiments, the one or more sites within the PTM binding pocket are experimentally-determined. In some embodiments, the PTM is negatively charged. The PTM can be any negatively charged PTM. In some embodiments, the PTM is phosphorylation. In some embodiments, the PTM is glycosylation. In some embodiments, the glycosylation is sialylation. In some embodiments, the amino acid that repels the PTM is a negatively-charged amino acid. Any kind of negatively-charged amino acid, naturally occurring or not, can be used in the methods provided herein. In some embodiments, the negatively-charged amino acid is aspartic or glutamic acid.

In some embodiments, the PTM is positively charged. The PTM can be any positively charged PTM. In some embodiments, the PTM is retinylidene Schiff base formation or arginylation. In some embodiments, the amino acid that repels the PTM is a positively-charged amino acid. Any kind of positively-charged amino acid, naturally occurring or not, can be used in the methods herein. In some embodiments, the positively-charged amino acid is lysine, arginine, or histidine.

In some embodiments, the amino acid that repels the PTM is a non-canonical amino acid. Any kind of non canonical amino acid, naturally occurring or not, can be used in the methods herein. In some embodiments, the non-canonical amino acid is phosphoserine, phosphotyrosine, p-azido-phenylalanine, benzoyl-phenylalanine, or acetyl-lysine.

In some embodiments, the amino acid that repels the PTM is introduced by a suppressor tRNA. Any kind of amino acid introduced in this way, naturally occurring or not, can be used in the methods herein. In some embodiments, such amino acid is selenocysteine or pyrrolysine.

In some embodiments, the PTM is hydrophobic. Any kind of hydrophobic PTM is amendable to the methods herein. In some embodiments, when the PTM is hydrophobic, the amino acid that repels the PTM is hydrophilic. Thus, in some embodiments, the amino acid that repels the PTM is hydrophilic. Any kind of hydrophilic amino acid, naturally occurring or not, can be used in the methods herein. In some embodiments, the hydrophilic amino acid is arginine, asparagine, aspartate, glutamine, glutamate, histidine, lysine, serine, or threonine.

In some embodiments, the PTM is hydrophilic. In some embodiments, when the PTM is hydrophilic, the amino acid that repels the PTM is hybrophobic. Thus, in some embodiments, the amino acid that repels the PTM is hydrophobic. Any kind of hydrophobic amino acid, naturally occurring or not, can be used in the methods herein. In some embodiments, the hydrophobic amino acid is glycine, alanine, valine, leucine, isoleucine, proline, phenylalanine, methionine or tryptophan.

Any method known in the art can be used to introduce the amino acid that repels the PTM. In some embodiments, the amino acid that repels the PTM is introduced by site mutagenesis.

In some embodiments, a context sequence adjacent to the PTM binding pocket site is randomized to generate a library of non-PTM binding antibodies. In some embodiments, the context sequence comprises 3-15 residues upstream or downstream to the PTM site. Any method of site-directed mutagenesis known in the art can be used to randomize the one or more regions outside the PTM binding pocket that binds to the context sequence adjacent to the PTM site. For example, error-prone PCR, site directed mutagenesis by primer extension, inverse PCR, kunkel-based mutagenesis, cassette mutagenesis, whole plasmid mutagenesis, AXM mutagenesis, error-prone rolling circle amplification (RCA), or in vivo site-directed mutagenesis can be used to introduce randomization to the context sequence to generate the antibody library. In some embodiments, the one or more regions outside the PTM binding pocket that bind to the context sequence are randomized by error-prone rolling circle amplification (RCA).

In some embodiments, the one or more regions outside the PTM binding pocket that bind to the context sequence are randomized without altering the PTM binding pocket.

Any kind of library can be used for displaying the candidate pan-PTM binding antibodies or non-PTM binding antibodies. For example, phage display, bacterial display, yeast display, ribosome display and/or mRNA display can be used. In some embodiments, the library comprising candidate pan-PTM binding antibodies or non-PTM binding antibodies is a phage display library.

In some embodiments, the library comprising candidate pan-PTM binding antibodies or non-PTM binding antibodies has a diversity of at least 10⁷, 10⁸, 10⁹, 10¹⁰, 10¹¹, or 10¹².

Any method known in the art can be used to screen the antibody library. For example, phage display or a micro-emulsion screening approach can be used. In some embodiments, the step of screening the library comprises whole cell panning. In some embodiments, the whole cell panning is emulsion based.

In some embodiments, the method further comprises a step of validating the identified pan-PTM binding antibodies or non-PTM binding antibodies. Any method known in the art can be used for validating the pan-PTM binding antibodies or non-PTM binding antibodies. For example, ELISA and/or functional assays can be used for antibody validation. In some embodiments, the validating step is high throughput.

As discussed above, and in more detail below, the PTM of the methods herein can be any kind of PTM, and validating of the antibodies can be performed by various methods, for example through high throughput validation or with a functional assay. In some embodiments, the PTM is phosphorylation and the high throughput validating step involves the use of a cell line that incorporates phospho-serine or phospho-tyrosine into suppressible amber (UAG) stop codons, thereby producing phosphorylated proteins for validating pan-PTM binding antibodies or non-PTM binding antibodies.

In the methods described herein, any kind of cell can be used. For example, the cell can be an archaeal cell, prokaryotic cell, bacterial cell, fungal cell, or eukaryotic cell. In some embodiments, the cell line is E. coli. In some embodiments, cell line is an insect cell line.

In some embodiments, the identified pan-PTM binding antibodies or non-PTM binding antibodies are validated by a functional assay. In some embodiments, the step of validating the pan-PTM binding antibodies or non-PTM binding antibodies comprises converting scFv to IgG in order to perform the validation. Any method in the art can be used for converting the scFv to IgG. For example, one method of converting the scFv to IgG includes cloning the variable heavy and light chain genes into pMINERVA vectors and expressing the vectors by transient transfection of CHO cells.

In further embodiments, the step of validating comprises determining if the identified pan-PTM binding antibodies or non-PTM binding antibodies are steric inhibitor antibodies against PTM.

In some embodiments, a method of generating a steric inhibitory antibody against an enzyme is provided, said method comprising providing an antibody that specifically recognizes a PTM on an enzyme of interest; identifying a PTM binding pocket of the antibody; generating a library comprising candidate pan-PTM binding antibodies by randomizing one or more regions inside or outside the PTM binding pocket that bind to the PTM site on the enzyme; screening the library against the enzyme of interest and selecting the antibodies that recognize the PTM site in the absence of PTM modification; and selecting steric inhibitory antibodies by performing an enzymatic activity assay.

In some embodiments, a method of generating a steric inhibitory antibody against an enzyme is provided, said method comprising engineering a PTM site on an enzyme of interest; providing an antibody that specifically recognizes the engineered PTM site on the enzyme eof interest; identifying a PTM binding pocket of the antibody; generating a library comprising candidate pan-PTM binding antibodies by randomizing one or more regions inside or outside the PTM binding pocket that bind to the engineered PTM site on the enzyme; screening the library against the enzyme of interest that does not have the engineered PTM site and selecting the antibodies that recognize the antibody in the absence of the engineered PTM site; and selecting steric inhibitory antibodies by performing an enzymatic activity assay.

In some embodiments, the PTM site is a naturally occurring PTM site. In some embodiments, the PTM site is an engineered PTM site. In some embodiments, the engineered PTM site is introduced into the enzyme of interest by site-specific mutagenesis. Various manners known in the art can be used to produce the engineered PTM site. In some embodiments, the site-specific mutagenesis comprises a point mutation, a series of point mutations, a deletion or an insertion. In some embodiments, the engineered PTM site is introduced by one or more amino acid substitutions in the enzyme of interest. In some embodiments, the engineered PTM site is introduced by insertion of one or more amino acids into the enzyme of interest. In some embodiments, the one or more amino acids inserted into the enzyme of interest comprises about 1, 2, 3, 4, 5, 6, 7, 8, 9, 10, 11, 12, 13, 14, 15, 16, 17, 18, 19, 20, 21, 22, 23, 24 or 25 amino acids. In some embodiments, the one or more amino acid substitutions can be 1, 2, 3, 4, 5, 6, 7, 8, 9, 10, 11, 12, 13, 14, 15, 16, 17, 18, 19, 20, 21, 22, 23, 24 or 25 amino acid substitutions.

In some embodiments, a pan-PTM binding antibody or a non-PTM binding antibody is generated according to a method of any one of the preceding disclosures.

In one aspect, the present disclosure provides, among other things, a pan-PTM-binding antibody library comprising a plurality of antibodies derived from a pre-existing antibody that specifically recognizes a PTM on a peptide or protein of interest, wherein the plurality of antibodies comprise a PTM binding pocket and one or more randomized regions that bind to a context sequence adjacent to the PTM site. In some embodiments, the PTM binding pocket comprises a non-charged amino acid at one or more sites that are determined to interact with the PTM.

Any non-charged amino acid, naturally occurring or not, can be used in the methods herein. In some embodiments, the non-charged amino acid is alanine or glycine.

The PTM in the methods provided herein can be any PTM. For example, the PTM can be any kind of acetylation, amidation, deamidation, prenylation (such as farnesylation or geranylation), formylation, glycosylation, hydroxylation, methylation, myristoylation, nitrosylation, phosphorylation, sialylation, sulphation, polysialylation, ubiquitination, SUMOylation, NEDDylation, ribosylation, sulphation, or any combinations thereof. In some embodiments, the PTM is phosphorylation. In some embodiments, the PTM is glycosylation. Any kind of glycosylation is congruent with the methods described herein. In some embodiments, the glycosylation is sialylation.

In one aspect, the present disclosure provides, among other things, a non-PTM-binding antibody library comprising a plurality of antibodies derived from a pre-existing antibody that specifically recognizes a PTM on a peptide or protein of interest, wherein the plurality of antibodies comprise a PTM binding pocket and one or more randomized regions that bind to a context sequence adjacent to the PTM site, wherein the PTM binding pocket comprises an amino acid that repels the PTM at one or more sites that are determined to interact with the PTM.

In some embodiments, the PTM is negatively charged. Any negatively charged PTM is congruent with the methods described herein. In some embodiments, the non-PTM-binding antibody library comprises a plurality of antibodies derived from a pre-existing antibody that specifically recognizes a PTM. In some embodiments, the PTM is phosphorylation. In some embodiments, the PTM is glycosylation. In some embodiments, the the glycosylation is sialylation.

In some embodiments, the method of generating non-PTM-binding antibodies includes introducing an amino acid that repels the PTM at one or more sites in the PTM binding pocket of the antibody. In some embodiments, the amino acid that repels the PTM is a negatively-charged amino acid. Any negatively-charged amino acid, naturally occurring or not, can be used in the methods described herein. In some embodiments, the negatively-charged amino acid is aspartic or glutamic acid.

In some embodiments, the PTM is positively charged. Any positively charged PTM is congruent with the methods described herein. In some embodiments, the PTM is retinylidene Schiff base formation or arginylation. In some embodiments, the amino acid that repels the PTM is a positively-charged amino acid. Any positively-charged amino acid, naturally occurring or not, can be used in the methods described herein. In some embodiments, the positively-charged amino acid is lysine, arginine, or histidine.

In some embodiments, the PTM is hydrophobic. Any hydrophobic PTM is congruent with the methods described herein. In some embodiments, the amino acid that repels the PTM is hydrophilic. Any hydrophilic amino acid, naturally occurring or not, can be used with the methods described herein. In some embodiments, the hydrophilic amino acid is arginine, asparagine, aspartate, glutamine, glutamate, histidine, lysine, serine, or threonine.

In some embodiments, the PTM is hydrophilic. Any hydrophilic PTM is congruent with the methods described herein. In some embodiments, the amino acid that repels the PTM is hydrophobic. Any hydrophobic amino acid, naturally occurring or not, can be used in the methods described herein. In some embodiments, the hydrophobic amino acid is glycine, alanine, valine, leucine, isoleucine, proline, phenylalanine, methionine or tryptophan.

As described above, and in further details below, the PTM can be any PTM. In some embodiments, the PTM is phosphorylation. In the methods described herein, the pre-existing antibody can bind any phosphorylated residue. In some embodiments, the phosphorylated residue is Ser, Tyr, and/or Thr. Thus, in some embodiments, the PTM is phosphorylation and the pre-existing antibody binds phosphorylated Ser (pSer), phosphorylated Tyr (pTyr), and/or phosphorylated threonine (pThr). In some embodiments, the PTM site is pSer. In some embodiments, the PTM site is pTyr. In some embodiments, the PTM site is pThr. In some embodiments, the PTM site is pHis (phosphorylated histidine). In some embodiments, the PTM site is pArg (phosphorylated arginine). In some embodiments, the PTM site is pLys (phosphorylated lysine). In some embodiments, the PTM site is a combination of pSer, pTyr, pThr, pHis, pArg, and/or pLys. In some embodiments, the PTM site is a combination of pSer and pTyr. In some embodiments, the PTM site is a combination of pSer and pThr. In some embodiments, the PTM site is a combination of pTyr and pThr.

In some embodiments, the PTM-binding pocket comprises a CDR H2 region and/or a CDR L2 region. In some embodiments, the one or more randomized regions bind to a context sequence that comprises 3-15 residues upstream or downstream of the PTM site.

In some embodiments, the one or more randomized regions of the pan-PTM-binding or non-PTM-binding antibody library comprise randomized CDR H3, or L3. In some embodiments, the one or more randomized regions comprise randomized CDR H1, L1, H3 and/or L3. In some embodiments, the one or more randomized regions comprise randomized framework regions.

In some embodiments, the PTM binding pocket of the pan-PTM-binding or non-PTM-binding antibody library comprises a CDR H2 region, and the one or more randomized regions comprise randomized CDR H3, CDR L3, and/or CDR L2.

In some embodiments, the one or more randomized regions of the pan-PTM-binding or non-PTM-binding antibody library are generated by site-directed mutagenesis.

In some embodiments, the one or more randomized regions of the pan-PTM-binding or non-PTM-binding antibody library are generated by error-prone RCA.

In some embodiments, the one or more randomized regions of the pan-PTM-binding or non-PTM-binding antibody library are generated by alanine and/or histidine scanning.

In some embodiments, the library is selected from the group consisting of a phage display library, a bacterial display library, a yeast display library, a ribosome display library, and an mRNA display library

In some embodiments, the library comprises a plurality of antibodies that are selected from antibody fragments. In some embodiments, the library comprises a plurality of antibodies that are selected from scFvs, Fabs, Fab′s, Fvs, or IgGs. In some embodiments, the library is a scFv library. In some embodiments, the scFv library is an M13 scFv library. In some embodiments, the library has a diversity of greater than 10⁷, 10⁸, 10⁹, 10¹⁰, 10¹¹, 10¹².

The present disclosure provides, among other things, a method of producing an antibody to a peptide of interest, comprising: providing a modified peptide of interest modified to include a negatively charged amino acid, screening the modified peptide against an antibody library biased towards the negatively charged amino acid, isolating one or more antibodies that bind to the modified peptide of interest; and determining the binding of the one or more antibodies to the peptide of interest without the modification, thereby identifying an antibody that binds to the peptide of interest.

The present disclosure provides, among other things, a method of producing an antibody to a peptide of interest, comprising: providing a modified peptide of interest modified to include a negatively charged amino acid, screening the modified peptide against an antibody library biased towards the negatively charged amino acid, isolating one or more antibodies that bind to the modified peptide of interest; generating a library of clonotypes of the one or more antibodies by affinity maturation; screening the library of the clonotypes against the peptide of interest without the modification, thereby identifying an antibody that binds to the peptide of interest.

In embodiments, the negatively charged amino acid is a phosphorylated amino acid. In embodiments, the phosphorylated amino acid is a phosphoserine (SEP) or a phosphotyrosine. In embodiments, the phosphorylated amino acid is a phosphoserine (SEP). In embodiments, the phosphorylated amino acid is a phosphotyrosine. In embodiments, the negatively charged amino acid is an aspartic acid or glutamic acid. In embodiments, the negatively charged amino acid substitutes a naturally-occurring amino acid within the peptide of interest. In embodiments, the antibody library is a phospho-biased antibody library. In embodiments, the phospho-biased antibody library is selected from a phage display library, a bacterial display library, a yeast display library, a ribosome display library, or an mRNA display library. In embodiments, the antibody library is a phage display library. In embodiments, the antibody library comprises a plurality of antibodies that are selected from antibody fragments. In embodiments, the antibody library comprises a plurality of antibodies that are selected from scFvs, Fabs, Fab′s, Fvs, or IgGs. In embodiments, the antibody library is a scFv library. In embodiments, the scFv library is a M13 scFv library. In embodiments, the antibody library has a diversity of about or greater than 10⁷, 10⁸, 10⁹, 10¹⁰, 10¹¹, or 10¹².

In embodiments, the antibody library has a diversity of about or less than 10^{13,} 10¹², 10¹¹, or 10¹⁰. In embodiments, the method further comprises affinity maturation of the antibody that binds to the peptide of interest. In embodiments, the affinity maturation is performed by directed evolution. In embodiments, the affinity maturation comprises a step of randomizing sequences outside the CDR-H2 and/or the CDR-L2 region of the one or more antibodies that bind to the modified peptide. In embodiments, the affinity maturation comprises error-prone PCR mutagenesis. In embodiments, each of the screening steps is performed on a whole cell, cell fragment, or isolated protein. In embodiments, each of the screening steps comprises whole-cell panning. In embodiments, the whole cell panning is emulsion based. In embodiments, the emulsion based screening is Delayed Emulsion Infectivity (DEI). In embodiments, the peptide of interest is based on an epitope of a protein of interest. In embodiments, the epitope is in an ectodomain of the protein of interest. In embodiments, the epitope is a linear epitope. In embodiments, the epitope is a conformational or discontinuous epitope. In embodiments, the peptide of interest comprises about between 5 and 30 amino acids. In embodiments, the peptide of interest comprises about between about 10 and 20 amino acids. In embodiments, the peptide of interest comprises a sequence around every 3^{rd}, 4^{th}, 5^{th}, 6^{th}, 7^{th}, 8^{th}, 9^{th}, or 10^{th} amino acid in an external domain of the protein of interest. In embodiments, the peptide of interest comprises a sequence around every 5^{th} amino acid in an external domain of the protein of interest. In embodiments, the peptide of interest does not include a glycosylation site. In embodiments, the protein of interest is a cell surface receptor. In embodiments, the cell receptor is a G-protein coupled receptor (GPCR), enzyme-coupled receptor, or a ligand-gated ion channel receptor. In embodiments, the cell receptor is a Leptin Receptor, Glucagon Receptor, Insulin Receptor, CXCR4, NTSR1, NTSR2 or a Receptor Tyrosine Kinase. In embodiments, the method further comprises a step of testing if the antibody binds the protein of interest. In embodiments, the method further comprises a step of testing if the antibody inhibits a function of the protein of interest. In embodiments, the function of the protein of interest is inhibited by the antibody by competitive inhibition. In embodiments, the function of the protein of interest is inhibited by the antibody by non-competitive inhibition. In embodiments, the non-competitive inhibition is allosteric inhibition. In embodiments, wherein the method further comprises a step of testing if a function of the protein of interest is augmented by the antibody.

The present disclosure provides, among other things, a method of producing an antibody that inhibits a protein of interest comprising: synthesizing a peptide based on a sequence from a protein of interest, wherein the sequence is modified to include a negatively-charged amino acid, identifying an antibody that binds to the peptide according to a method of any one of the preceding claims; and testing if the antibody inhibits a function of the protein of interest. In embodiments, the method further comprises a step of determining if the antibody sterically inhibits the protein of interest.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

FIG. 1 is a series of schematics that show one embodiment of a method to derive phosphostatus-specific antibodies (phophostatus-specific antibody clonotypes). The schematics illustrate a method to develop antibody clonotypes that differ only in their ability to detect the PTM status (e.g. phosphostatus; phosphostatus specific antibody clontypes) of specific context sequences. The method can be further used to derive antibodies against any given peptide, by first producing a phospho-derivative to direct binding, and then affinity mature this derivative to increase affinity to the context sequence.

FIG. 2 is a flow diagram that depicts the development of antibodies that are inhibitors of cell surface proteins. FIG. 2, panel A depicts the synthesis of 10 to 20-mer peptides composed of overlapping portions of an ectodomain of the receptors of interest (depicted is a GPCR). In this flow diagram, the 10-20mer peptides are synthesized with a central phospho-serine (SEP) replacing the naturally occurring amino acid. FIG. 2, panel B depicts the phage display stage in the process of developing antibody allosteric inhibitors to the cell surface protein of interest. In this flow diagram, phage display is used to identify phospho-specific scFv hits. FIG. 2, panel C of the flow diagram shows that the identified hits are mutagenized, and undergo maturation (e.g. discovery matured (“DisMAT”) and/or affinity maturation (“AffMAT”) against the native sequence peptide. FIG. 2, panel D of the flow diagram indicates that IgG will be purified, followed by testing of the purified IgGs in cell-based assays.

FIG. 3 shows a schematic of the human thrombin protein and a series of graphs (FIG. 3, panels (a)-(d)) that depict the activity of antibody allosteric inhibitors developed against human thrombin protein in accordance with the methods described herein.

FIG. 4, panels A-F, is a series of schematics (panels A-D and F) that depict a model of ScFv in complex with a phospho-peptide based on computational methods and experimental data from alanine scanning (panel E). FIG. 4, panel A, shows an overlay of top 200/2000 calculated structure decoys using Rosetta protein modelling software's Rosetta Ab protocol. FIG. 4, panel B, shows that the two top scored structures displayed similar CDR conformations. FIG. 4, panel C, shows that the electrostatic contour of the structure model displayed a deep negative charged groove connecting to H2. FIG. 4, panel E, shows alanine scanning mutagenesis results. The residues important for affinity, as shown in FIG. 4, panel E, form part of the groove as shown in FIG. 4, panel D. The positive charged half of the phospho-antigen (MARRPRHSIYS(phos)SDEDDEDFE) was manually modelled into the groove and refined using Rosetta FlexiPepDock (FIG. 4, panel F). The results show that the left most arginine is not contacting the protein.

FIG. 5, panels A-D, is a series of schematics (panels A-B), a bar graph (panel C), and a table (panel D) that shows the design and analysis of phosphate-specific libraries. Data indicate that a single residue substitution can switch pSer binding to pTyr binding. The structural difference is illustrated in FIG. 5, panels A and B. The mutated residues are labelled. The H3 and H2 CDR loops are labelled and the contacting residues are shown as sticks. FIG. 5, panel C, is a bar graph that shows ELISA results against a panel of peptides with anti-Myb phosphorylated serine (pSer)-specific scFv (AXM1293) and its mutants. FIG. 5, panel D, shows libraries in which L2, H3 and L3 were simultaneously randomized, and screened against different phospho-targets. The screening against the different phospho-targets was performed using a library in which CDR-H2 recognizes both pSer and pTyr and the unique hits numbers were compared with a traditional library approach.

FIG. 6 is a series of schematics that shows a modified pMINERVA scFv to IgG system. The scFv Abs are encoded on a pMINERVA phagemid pDonor vector as a gp3-fusions and are screened in a phage display biopanning procedure. Following the screen a phagemid encoding an scFv with the desired biophysical properties is identified. This phagemid is transduced into an E. coli strain expressing phiC31 integrase and harboring, as one example, an IgG acceptor vector (pAcceptor). The product of the recombination event introduces the CH gene and polyadenylation signal site adjacent to the 3′ end of the VH gene. Furthermore, the recombination event introduces both a mammalian promoter and functional protein initiation site 5′ to the VL gene. The linker between the VH and VL domains of the scFv is composed of a phiC31 36-bp attP site that is able to function as both a: i) peptide linker between the heavy and light variable domains, and ii) 36-bp functional substrate for phiC31 integrase. [Figure legend: Pmam, mammalian promoter; Pyeast, yeast promoter; Pcmv, CMV promoter; 5′ss and 3′ss, splice signals; VL, variable section of the light chain; VH, variable section of the heavy chain; gp3, phage M13 gene3 product; PE.coli, E.coli promoter; CH or Fc, constant region of the heavy chain; attB, attP, substrates for an integrase gene; attR and attL, products of an integrase gene; polyA, polyadenylation sequence; CamS, CamR, chloramphenicol resistance gene without and with a promoter, respectively; TCRzeta, T-cell receptor zeta; CAR-T, chimeric antigen receptor; Prosplice, Procat, dual-function promoter-types; IRES, internal ribosome entry site; RBS, ribosome binding site].

FIG. 7 is a series of schematics that show AXM mutagenesis for affinity maturation library construction approach. The pool of oligonucleotides is amplified under error-prone PCR conditions using a reverse primer containing multiple phosphorothioate linkages on its 5′ terminus. The resulting double-stranded DNA is treated with T7 exonuclease to selectively degrade the unmodified strand of the dsDNA molecule. The resulting single-stranded DNA, or ‘megaprimer’, is then annealed to the uracilated, circular, single-stranded phagemid DNA and used to prime in vitro synthesis by DNA polymerase. The ligated, heteroduplex product is then transformed into E. coli AXE688 cells, where the uracilated strand is cleaved in vivo by uracil N-glycosylase, favoring survival of the newly synthesized, recombinant strand containing the megaprimer. Also, Eco29kI is used to eliminate any clones that are not fully recombinant.

FIG. 8 is a series of schematics that show phage display. Phage display is used for the high-throughput screening of protein interactions. In the case of M13 filamentous phage display, the DNA encoding the protein or peptide of interest is ligated into the pIII gene, encoding the minor coat protein. The phage gene and insert DNA hybrid is then transduced into E. coli TG1. By immobilizing a relevant protein target to the surface of a microtiter plate well or bead, a phage that displays an Ab that binds to one of those targets on its surface will remain while others are removed by washing. Those that remain can be eluted, used to produce more phage (by bacterial infection with helper phage) and so produce a phage mixture that is enriched with relevant (i.e. binding) phage. Elution can be done combining low-pH elution buffer. The repeated cycling of these steps is referred to as ‘panning’, in reference to the enrichment of a sample of gold by removing undesirable materials. Phage eluted in the final step can be used to infect a suitable bacterial host, from which the phagemids can be collected and the relevant DNA sequence excised and sequenced to identify the relevant, interacting proteins or protein fragments.

FIG. 9 is a series of schematics that show a phage micro-emulsion screening method. In this screening approach, E. coli infected with a library of bacteriophage M13 encoding scFv variants, are compartmentalized with antigen-coated beads in a water-in-oil emulsion to give, on average, approximately 1 bead and 1-10 infected-bacterial calls per compartment. Consequently, in each compartment, multiple copies of the recombinant phage are produced, some of which may bind to the antigen-coated bead. The emulsion is broken and the microbeads, along with any bound phage, are isolated. The beads are then incubated with anti-M13 IgG Ab coupled to FITC. Hence, antigen-coated beads with bound phage become labelled with multiple fluorescein molecules. These beads can then be enriched (together with the phage attached to them) by flow cytometric sorting.

FIG. 10, panels a-b, is a series of schematics that show the use of in vivo restriction and transformation with saturating plasmid DNA to produce high-titer fully recombinant antibody libraries. FIG. 8, panel a, shows in vivo restriction using AXE688 strain in producing recombinant libraries. Parental plasmids carrying Eco29kI sites within the complementarity determining regions (CDRs) are cleaved by the Eco29kI enzyme expressed in the AXE688 [=TG1 (eco29KIR, eco29kIM)] cells. FIG. 10, panel b, depicts in vivo cell selection using saturating DNA. The selectivity of the cells can be exploited by using super-saturating concentrations of plasmid DNA to generate large libraries of fully recombinant clones. Competent cells within the electro-competent cell aliquot can take up multiple plasmids under conditions which are DNA saturating relative to the number of competent cells in the mix. Using AXE688, the cells take up multiple plasmids and restrict parental DNA retaining Eco29kI sites in vivo and thereby result in transformed cells with a higher proportion of totally-recombinant clones.

FIG. 11, panels A-C depict a series of schematics (FIG. 11, panel A) and graphs (FIG. 11, panels B and C) that demonstrate the isolation of anti-idiotype antibodies specific to the CDRH3 of a selected antibody. FIG. 11, panel D depicts a series of schematics and graphs that show representative examples of isolated antibodies that inhibit the following enzymes: thrombin, amylase and sortase. The schematics indicate the area in which the isolated antibody binds to the enzyme target. The graphs show inhibition by the antibodies under various conditions.

FIG. 12, panels A-D depict a series of micrographs and graphs that are representative examples of antibodies that have been made using phage-derived IgG antibodies as described herein. FIG. 12, panel A depicts the results of immunocytochemistry staining of an antibody specific for Myelin Basic Protein in SK-N-SH (human neuroblastoma cell line cells). FIG. 12, panel B depicts a Western blot using an antibody specific for Histone H2B, performed on HeLa cells. FIG. 12, panel C depicts a flow cytometry graph in which the flow cytometry experiment used sc-Fv E1 antibody incubated with a cell line that overexpressed the NTSR1 GPCR. FIG. 12, panel D is a graph that shows the results of a chromatin immunoprecipitation assay (ChIP) using an antibody specific for Histone H2B.

FIG. 13, panels (a)-(c) shows a series of schematics representing the identification and validation of modification-specific clonotypes (“MSC”) antibody pairs in Western analysis. FIG. 13, panel (a) is a schematic that shows that shows a diagram of Directed Phospho-Ab Clonotypes (“DPAC”). DPAC is a platform for the isolation and validation of new phospho-specific antibodies and matching antibody pairs that recognize the same sequence, free of modification. FIG. 13, panel (b) is a schematic that illustrates molecular evolution of phospho-specific phage antibody hits through the use of discovery maturation (“DisMat”). FIG. 13, panel (c) is a schematic that shows validation of the isolated, converted IgG in E. coli.

FIG. 14 is a schematic that shows the use of animal-derived phospho-specific antibodies. Data obtained from studies using animal-derived phospho-specific antibodies indicate a 66% success rate against 6 test RAbMab clones in finding phospho-independent or serine specific antibodies. Directed Phospho-Ab Clonotypes 3 (“DPAC3”) refers to a platform for the conversion of existing anti-phospho-modified recombinant monoclonal antibodies into unmodified and phospho-independent clonal derivatives.

FIG. 15, panel A and panel B are a series of graphs that show the discovery and Discovery Maturation (“DisMat”) of anti-akt473.

FIG. 16A is a schematic that shows delayed emulsion infectivity (“DEI”): recombinatorial biopanning. DEI is an emulsion based screening assay.

FIG. 16B is a bar graph that shows the effect of growth temperature on infection by M13 in the DEI assay. E. coli TG1 cells were grown at 20° C., 24° C., or 37° C. to OD600=0.4. The cells were then incubated with phage, washed (to remove unbound phage), and plated at 37° C. for ampicillin transduction. The number of ampicillin-resistant colonies under each condition was counted and plotted.

FIG. 17, panels A and B are schematics that show a bacterial display system used in an embodiment of DEI. FIG. 17, panel (A) shows a schematic image of the Lpp-OmpA bacterial display system. FIG. 17, panel (B) shows a schematic of an expression construct for antigen display containing E. coli promoter (lac), Lpp-OmpA fusion protein, Tev protease cleavage site, and antigen fragment. The promoter, display protein (Lpp-OmpA), and antigen sequences are each flanked by restriction sites to facilitate cloning of alternate sequences. (Right) Integration by phiC31 Integrase. In the E. coli host, the phiC31 integrase protein recombines the attP and attB sequences of the two vectors and thereby produces a single, chimeric molecule composed of both homing and donor vector sequences in a pre-defined orientation. The product of the recombination event fuses the VH to the CH gene. Furthermore, the recombination event introduces both a mammalian promoter and functional protein initiation site 5′ to the VL gene. Of special note, the linker between the VH and VL domains of the scFv is composed of phiC31 36-bp attP site that is able to function as both: i) a peptide linker between the heavy and light variable domains, and ii) a 36-bp functional substrate for phiC31 integrase. [Figure legend: PCMV, CMV promoter; mSigP, mammalian signal peptide; bSigP, bacterial signal peptide; ss, splice signal; VL, variable section of the light chain; VH, variable section of the heavy chain; gp3, phage M13 gene3 product; PSacR, B. subtilis SacR promoter; Ptac, chimeric trp and lac promoter; Fc, constant region of the heavy chain; attB, attP, substrates for the phiC31 integrase gene; attR and attL, products of the phiC31 integrase gene; loxP, substrates for the cre protein; polyA, polyadenylation sequence].

FIG. 18, panels A and B are a series of graphs that show results from a multiplex of 18 separately displayed peptides on the surface of E. coli. Shown are an analysis of the occurrence of over 1 million antibody Next Generation Sequencing (“NGS”) reads for the first 10 peptides. FIG. 18, panel A shows NGS analysis of phage hits. Graphed as a function of the occurrence of a specific antibody sequence to the target N vs the entire NGS set (minus the target N). FIG. 18, panel B shows an explanation of the three “classes” or groupings of NGS reads. As shown in FIG. 18, panel B, the three classes or groupings are (i) enriched Targetl-specific scFvs; (ii) non-specific scFvs; and (iii) enriched all-other scFvs.

FIG. 19 is a schematic that shows validation of Modification-Specific Clonotypes (“MSC”) antibodies against a protein using an orthologous antibody in a Western Analysis.

### DEFINITIONS

In order for the present invention to be more readily understood, certain terms are first defined below. Additional definitions for the following terms and other terms are set forth throughout the specification.

Affinity regent: As used herein, the term “affinity reagent” is any molecule that specifically binds to a target molecule, for example, to identify, track, capture or influence the activity of the target molecule. The affinity reagent identified or recovered by the methods described herein are “genetically encoded,” for example an antibody, peptide or nucleic acid, and are thus capable of being sequenced. The terms “protein,” “polypeptide,” and “peptide” are used interchangeably herein to refer to two or more amino acids linked together.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” or “Ab” or “Abs” refers to immunoglobulin molecules and immunologically active portions of immunoglobulin (Ig) molecules, i.e., molecules that contain an antigen binding site that specifically binds (immunoreacts with) an antigen. By “specifically binds” or “immunoreacts with” tt is meant that the antibody reacts with one or more antigenic determinants of the desired antigen. Antibodies include, antibody fragments. Antibodies also include, but are not limited to, polyclonal, monoclonal, chimeric dAb (domain antibody), single chain, F_{ab}, F_{ab′}, F_{(ab′)2} fragments, scFvs, and F_{ab} expression libraries. An antibody may be a whole antibody, or immunoglobulin, or an antibody fragment.

The recognized immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (of about 25 kDa or about 214 amino acids) comprise a variable region of about 110 amino acids at the NH2-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

Antigen binding site: As used herein, the term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Anti-idiotype antibody: As used herein, “anti-idiotype antibody” means an antibody that specifically binds to the antigen-binding site of another antibody and, therefore, is specifically bound by the other antibody. The anti-idiotype antibody can mimic the epitope normally recognized by another antibody. An idiotype is the genetically determined variation of structures in the variable regions of immunoglobulins. The precise genetic basis of idiotype variability has only been partially explained. However, idiotype variation involves the amino acid sequence and protein structure (so-called determinants) especially in the area of the antigen-binding site, also referred to as the idiotope. The term “idiotype” designates the complete set of determinants of a variable region of an antibody molecule.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Clonotype: As used herein, the term “clonotype” refers to a set of antibodies that derive from the same ancestral B cell through somatic cell affinity-maturation.\

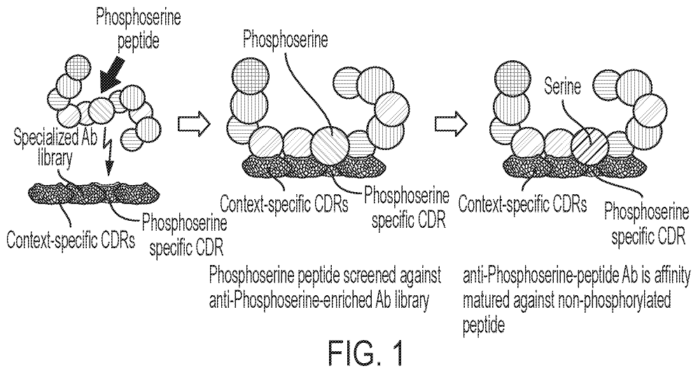

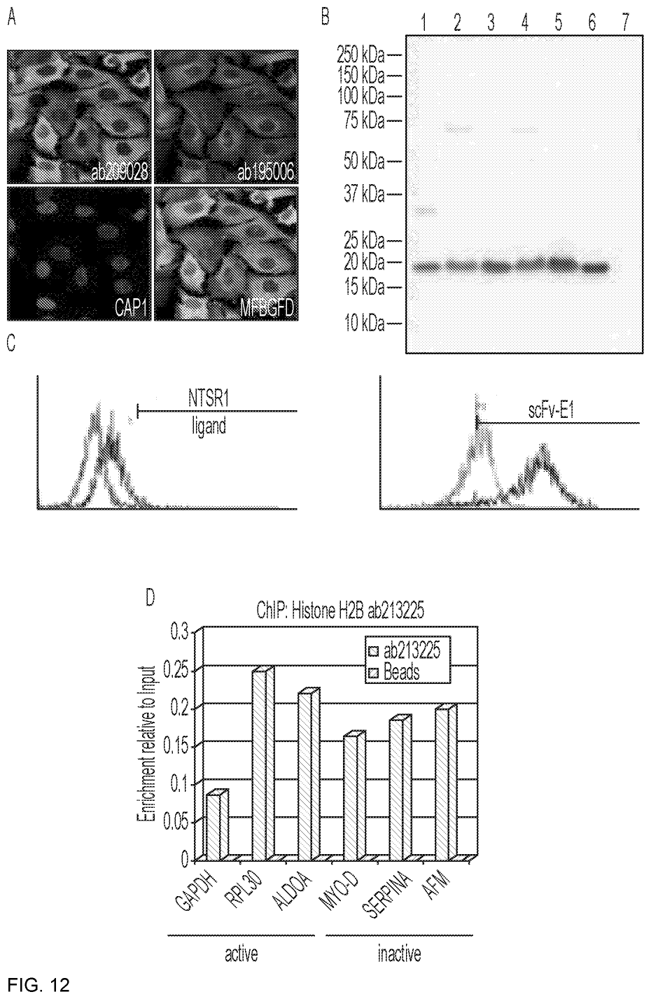

Directed Phosphostatus-specific antibody clonotypes 1 (“DPAC1”): As used herein “Directed Phosphostatus-specific antibody clonotypes 1” or “DPAC1” refers to a platform for the isolation and validation of new phospho-specific antibodies and matching antibody pairs that recognize the same sequence, free of modification (FIG. 1 and FIG. 13).

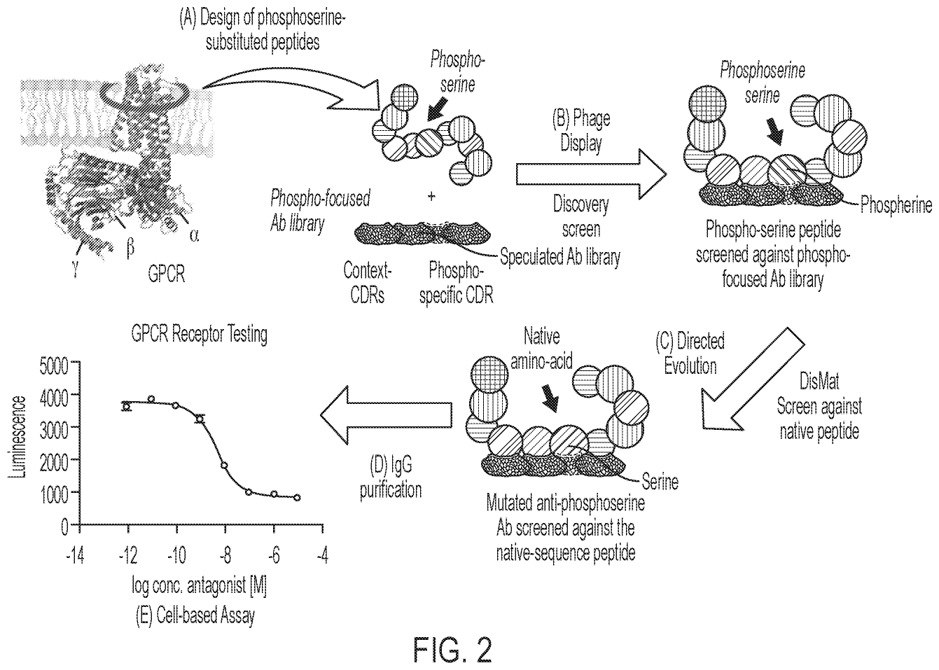

Directed Phosphostatus-specific antibody clonotypes 2 (“DPAC2”): As used herein “Directed Phosphostatus-specific antibody clonotypes 2” or “DPAC2” refers to a platform for the isolation of antibody inhibitors against a peptide or protein of interest. In embodiments, the peptide or protein of interest can be an enzyme. In embodiments, the inhibition is via steric inhibition. DPAC2 also refers to a platform for producing anti-idiotype antibodies (FIG. 2).

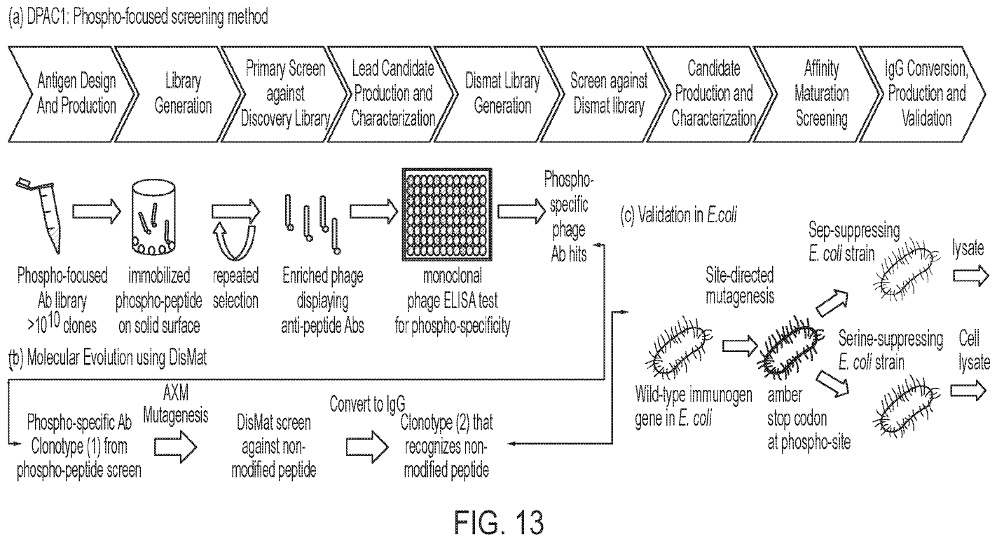

Directed Phosphostatus-specific antibody clonotypes 3 (“DPAC3”): As used herein “Directed Phosphostatus-specific antibody clonotypes 3” or “DPAC3” refers to a platform for the conversion of existing anti-phospho-modified recombinant monoclonal antibodies into unmodified and phospho-independent clonal derivatives (FIG. 14).

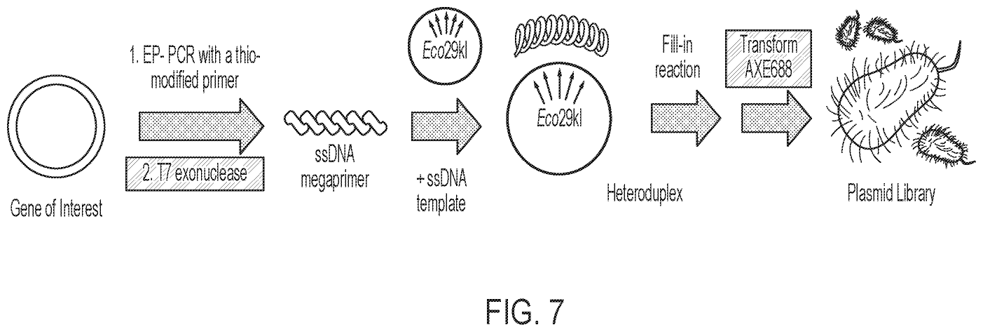

Discovery Maturation (“DisMat”): As used herein, “Discovery Maturation,” or “DisMat” refers to AXM error-prone PCR mutagenesis (FIG. 7) to produce random mutation libraries of the discovery hit clones and then biopan these newly-made libraries under discovery biopanning conditions where the antigen concentration is high and kept constant through 3-4 repeated rounds of panning. These altered-specificity clones, are referred to herein as “clonotype.” Binding can be further improved by also using Affinity Maturation (“AffMat”) directed evolution. For example, in AffMat the immunogen concentration is dropped 4-10 fold per each of 3-4 rounds of panning. AffMat employed in this manner allows for an off-rate selection protocol to increase the affinity of the clonotypes.

Epitope: As used herein, the term “epitope” includes any protein determinant capable of specific binding to an immunoglobulin, or fragment. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

Functional epitope: As used herein, the term “functional epitope” means the residues within the epitope that make energetic contributions to the binding interaction.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Immunological Binding: The term “immunological binding” refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein smaller K_{d} represents a greater affinity. Immunological binding properties of selected polypeptides can be quantified using methods well known in the art.

Molecular Display System: As used herein, the term “molecular display system” or “antibody library” is any system capable of presenting a library of potential affinity reagents to screen for potential binders to a target molecule or ligand. Examples of molecular display systems include phage display, bacterial display, yeast display, ribosome display and mRNA display. In some embodiments, phage display is used.

Modification-Specific Clonotypes: As used herein, the term “Modification-Specific Clonotypes,” or “MSCs,” are antibodies that differ in a few amino acids and bind differentially to the post-translational state of the same epitopes. “Few amino acids” means about 1, 2, 3, 4, 5, 6, 7, 8, 9, 10, 11, 12, 13, 14, 15, 16, 17, 18, 19 or 20 amino acids.

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Post-translational modification: The term, “post-translational modification,” as used herein generally refers to any modification of a peptide or a protein that occurs either during or after protein biosynthesis. For example, it includes the covalent addition of functional groups or proteins to a protein or peptide, such asproteolytic cleavage of regulatory subunits or degradation of entire proteins.

Post-translational modification site: The term, “post-translational modification site,” as used herein generally refers to one or more amino acid residues within a peptide, protein, or enzyme, that act as acceptors for any modification(s). The post-translational modification site can be either naturally occurring, or it can be engineered into a peptide, protein or enzyme of interest.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

scFv: A single chain Fv (“scFv”) polypeptide molecule is a covalently linked VH::VL heterodimer, which can be expressed from a gene fusion including VH- and VL-encoding genes linked by a peptide-encoding linker. (See Huston et al. (1988) Proc Nat Acad Sci USA 85(16):5879-5883). A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,132,405; and 4,946,778.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION OF CERTAIN EMBODIMENTS

Various aspects of the invention are described in detail in the following sections. The use of sections is not meant to limit the invention. Each section can apply to any aspect of the invention. In this application, the use of “or” means “and/or” unless stated otherwise.

The present disclosure provides, among other things, methods and compositions for generating antibodies that recognize, bind to, and or modulate epitopes having specific post-translational modifications. In some embodiments, the current disclosure describes a structure-based directed evolution approach for the production of post-translational modification specific antibodies. In some embodiments, the disclosure describes the generation of pan-post-translational modification antibodies, in which the antibodies bind to an epitope in the presence or absence of the post-translational modification. In some embodiments, the present disclosure provides methods of producing antibodies that inhibit a peptide or protein of interest. In embodiments, the antibodies are anti-enzyme steric inhibitors.

The generation of antibodies that target specific post-translational modifications of an epitope allows for the use of said antibodies in many aspects of research and allows for potential therapeutic uses of the same. For example, antibodies can be generated that bind to and modify a therapeutic protein target or other biological target. By “biological protein target” is meant anything within a living organism (e.g. a cell, protein, small molecule, RNA, DNA and the like) to which some other entity is directed and/or binds, wherein the binding changes the cell and/or living organism's physiology.

Recombinant antibodies, like single chain variable fragments (scFv), have many attractive attributes. They are renewable through overexpression in the appropriate heterologous host, they are easily stored and transferred as DNA, and they can be genetically engineered as fusions to various enzymes, fluorescent proteins, and epitope tags. Also useful, scFvs can be easily converted to IgGs. However, prior to the present disclosure, in vitro selection methods, such as phage display, yeast display, and ribosome display have thus far been inefficient at meeting the need for customizing post-translational modification specific antibodies.

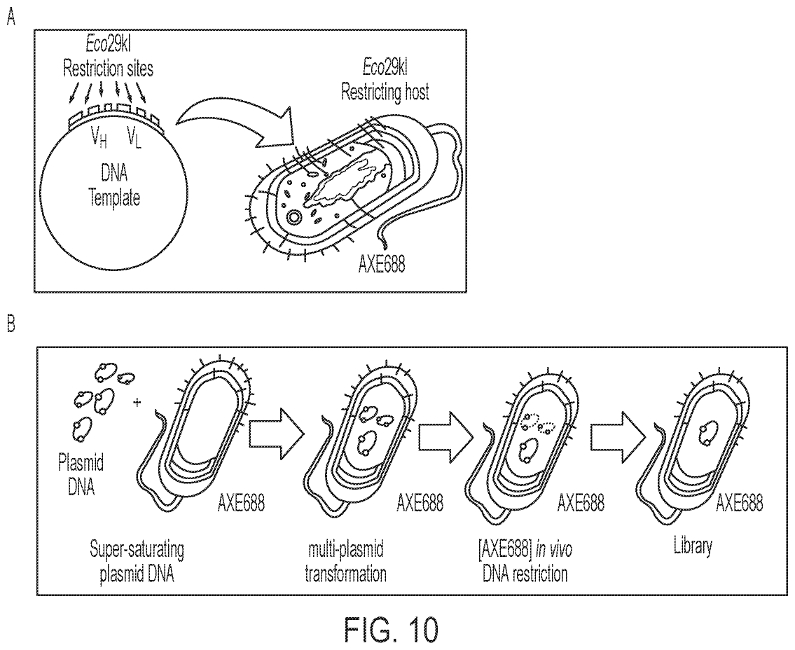

Several methods have previously been used to produce Ab diversity in vitro. These include cloning cDNAs of the immune regions (a native approach) of either immunized or non-immunized vertebrate cells (to create a naïve library), total synthesis of Ab CDR gene fragments with mixed-nucleotide synthesis, and a semi-synthetic approach whereby a framework gene is synthesized, and the diversity is generated by cloning a multitude of CDRs. A typical phage display library with a constant framework randomizes the 12 positions in 5 CDRS that are found to either: (i) vary the most in naturally-occurring Abs, or (ii) are projected to interact the most with a given antigen (“Ag”). The total potential diversity with these libraries is still substantially higher (>3.8×10²¹) than the diversity that can be sampled (typically 10¹⁰-10¹¹ with phage libraries), and not all amino acids at a given CDR position will yield a functional Ab. A good phage display library is generally >10¹¹ with >10% recombinant. Using methods provided herein for library production (FIG. 10), libraries can be made that are >10¹² and >85% recombinant. In some embodiments, as disclosed herein, a focused library for binding phosphorylated targets using 3 constant positions will be effectively 20³ (=8,000) times more efficient, valuable and practical over randomized libraries.

In some embodiments, instead of random mutagenesis of CDR residues, prior knowledge of post-translational modification antibody binding can be incorporated into the library randomization approach to produce a library with a higher frequency of relevant binders.

### Post-Translational Modifications

The methods described herein can be applied to any epitope having any kind of post-translational modification. The post-translational modification can be any post-translational modification. The post-translational modification can be negatively charged, positively charged, hydrophilic, and/or hydrophobic.

As non-limiting examples of post-translational modifications, the post-translational modification can be any kind of acetylation, amidation, deamidation, prenylation (such as farnesylation or geranylation), formylation, glycosylation, hydroxylation, methylation, myristoylation, phosphorylation, sialylation, polysialylation, ubiquitination, SUMOylation, NEDDylation, ribosylation, sulphation, or any combinations thereof.

In some embodiments, the post-translational modification can be acylation, including for example myristoylation, lipoylation, and/or palmitoylation. The post-translational modification can be, for example, isoprenylation, prenylation, farnesylation, geranylgeranylation, glypiation, addition of a Flavin moiety, heme c, phophopatetheinylation, retinylidene Schiff base formation, acylation, formylation, alkylation, methylation, amidation, arginylation, polyglutamylation, polyglycylation, butyrylation, gamma-carboxylation, glycosylation, malonylation, nitrosylation, hydroxylation, iodination, adenylylation, propionylation, pyroglutamate formation, S-glutathionylation, S-nitrosylation, S-sulfenylation, S-sulfinylation, S-sulfonylation, succinylation, sulfation, glycation, carbamylation, carbonylation, biotinylation, carbamylation, oxidation, pegylation, ISGylation, SUMOylation, ubiquitination, NEDDylation, Pupylation, citrullination, deamidation, eliminylation or any combinations thereof.

In some embodiments, the post-translational modification can include disulfide bridges, proteolytic cleavage, isoaspartate formation, racemization, protein splicing or any combinations thereof.

### Post-Translational Modification Site

In embodiments described herein, a PTM site can be any PTM site. The PTM site can be either naturally occurring or engineered. The engineered PTM site can be introduced into a peptide, protein or enzyme of interest by the substitution of one or more amino acids in the peptide, protein or enzyme of interest. Alternatively, or in addition to the preceding, an engineered PTM site can be introduced into a peptide, protein or enzyme of interest by inserting one or more amino acid residues into the peptide, protein, or enzyme of interest. The introduced engineered PTM site can be of any length. As a non-limiting example, the introduced PTM can comprise about 1-25, 1-50, 1-100 or 1-150 amino acids. In some embodiments, the introduced PTM comprises about 1, 2, 3, 4, 5, 6, 7, 8, 9, 10, 11, 12, 13, 14, 15, 16, 17, 18, 19, 20, 21, 22, 23, 24 or 25 amino acids. In some embodiments, the introduced PTM comprises about 1-10 (e.g. 1, 2, 3, 4, 5, 6, 7, 8, 9, or 10) or 1-15 (e.g. 1, 2, 3, 4, 5, 6, 7, 8, 9, 10, 11, 12, 13, 14, or 15) amino acids.

The introduced PTM can be inserted into the peptide, protein or enzyme of interest by any methods known in the art. For example, well-known methods of molecular cloning can be used to insert nucleotides coding for the introduced PTM site into a peptide, protein, or enzyme of interest. Alternatively, methods known in the art can be used to directly introduce an amino acid sequence corresponding to the desired PTM site into the peptide, protein or enzyme of interest.

In some embodiments, the engineered PTM can be introduced by the substitution of one or more amino acids present in a peptide, protein or enzyme of interest. For example, the engineered PTM site can be introduced by site mutagenesis or random mutagenesis. The site mutagenesis can be, for example, a point mutation, a series of point mutations (e.g. 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, or any amounts in between), a deletion or an insertion.

### Generation of PTM- and Pan-PTM Binding Antibody Library

Provided herein are methods to develop a structure-based directed evolution approach to produce antibodies that recognize a post-translational modification (PTM). In some embodiments, a method is provided of generating antibodies that recognize a PTM site comprising: providing an antibody that specifically recognizes a PTM on a peptide or protein of interest; identifying a PTM binding pocket of the antibody; introducing a negatively-charged or a non-charged amino acid at one or more sites within the PTM binding pocket of the antibody that are determined to interact with the PTM; generating a library comprising candidate pan-PTM binding antibodies by randomizing one or more regions outside the PTM binding pocket that bind to a context sequence adjacent to the PTM site; screening the library against the peptide or protein of interest without PTM, thereby identifying pan-PTM binding antibodies.

In one aspect, this disclosure provides methods of using phospho-amino acid-recognizing libraries that can be enriched by the incorporation of a phospho-specific-binidng hypervariable region. The anti-phosphorylation specificity can be used as a biophysical marker to “localize” the binding of a phospho-specific antibody to a specific site on a peptide, and thereby the protein also. Once the specific site is identified, the recombinant antibodies can be evolved to recognize the native sequence. In embodiments, this disclosure provides for the discovery and validation of anti-phosphoprotein antibodies and evolved antibodies that recognize high highly similar sequence.

In some embodiments, a method of generating antibodies includes providing an antibody that specifically recognizes a PTM on a peptide or protein of interest. The PTM can be any PTM. Exemplary PTM modifications are described above. Having a starting antibody that recognizes a specific post-translational modification allows for an analysis and identification of residues that directly interact with the post-translational modification. In some embodiments, the starting antibody is used for structural analysis and for a determination of the antibody PTM binding pocket.

In some embodiments, a PTM binding pocket of the starting antibody is identified. There are several methods in the art that allow for the identification of PTM binding pockets. Any suitable method can be used to identify the antibody PTM binding pocket. In some embodiments, one or more sites within the PTM binding pocket are structurally predicted. In some embodiments, one or more sites within the PTM binding pocket are experimentally determined.

In some embodiments, a non-charged amino acid is introduced at one or more sites within the PTM binding pocket of the antibody that are determined to interact with the PTM. By “introduced” is meant any one of the following: i) replacing an existing amino acid in its native position with another, seleted amino acid; ii) adding an additional, selected amino acid into a sequence at a selected position; or iii) removing a selected amino acid from a sequence and adding another amino acid to a distinct position in the sequence apart from the position of the original, removed amino acid. The non-charged amino acid can be introduced, for example, to about 1, 2, 3, 4, 5, 6, 7, 8, 9, 10, 11, 12, 13, 14, or 15 sites within the PTM binding pocket. In embodiments, the non-charged amino acid is introduced to 1 site within the PTM binding pocket. In embodiments, the non-charged amino acid is introduced adjacent to the PTM binding pocket. By “adjacent” is meant about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids removed from the PTM binding pocket. In some embodiments, the non-charged amino acid is alanine, isoleucine, leucine, methionine, phenylalanine, valine, proline or glycine. In some embodiments, the non-charged amino acid is alanine or glycine. The isolectric point (pI) of an amino acid can be used as a guide in selecting the non-charged amino acid (Table 1). Various methods can be used to introduce the non-charged amino acid into the PTM binding pocket of the antibody. Any method known in the art can be used. In some embodiments, the non-charged amino acid is introduced by site mutagenesis.

In some embodiments, a negatively-charged amino acid is introduced at one or more sites within the PTM binding pocket of the antibody that are determined to interact with the PTM. The negatively-charged amino acid can be introduced, for example, to about 1, 2, 3, 4, 5, 6, 7, 8, 9, 10, 11, 12, 13, 14, or 15 sites within the PTM binding pocket. In embodiments, the negatively-charged amino acid is introduced to 1 site within the PTM binding pocket. In embodiments, the non-charged amino acid is introduced adjacent to the PTM binding pocket. In some embodiments, the negatively-charged amino acid is a phosphorylated amino acid. In some embodiments, the negatively-charged amino acid is phosphoserine (SEP), phophotyrosine, aspartic acid or glutamic acid. In embodiments, the negatively-charged amino acid is phosphoserine (SEP). In embodiments, SEP is introduced so as to replace any one or more of the following amino acids: Ser (S), Gly (G), Glutamate (E), Aspartate (D), Phenylalanine (F), Tyrosine (Y), Leucine (L), and Isoleucine (I). The isolectric point (pI) of an amino acid can be used as a guide in selecting the negatively-charged amino acid (Table 1).

TABLE 1
Amino acid isolectric points pI

| Amino Acid | One Letter Abbreviation | pI (isoelectric point) |
|---|---|---|
| Alanine | A | 6.0 |
| Arginine | R | 10.76 |
| Asparagine | N | 5.41 |
| Aspartic Acid | D | 2.77 |
| Cysteine | C | 5.07 |
| Glutamic Acid | E | 3.22 |
| Glutamine | Q | 5.65 |
| Glycine | G | 5.97 |
| Histidine | H | 7.59 |
| Isoleucine | I | 6.02 |
| Leucine | L | 5.98 |
| Lysine | K | 9.74 |
| Methionine | M | 5.74 |
| Phenylalanine | F | 5.48 |
| Proline | P | 6.30 |
| Serine | S | 5.58 |
| Threonine | T | 5.60 |
| Tryptophan | W | 5.89 |
| Tyrosine | Y | 5.66 |
| Valine | V | 5.96 |

In some embodiments, the PTM can be any PTM. In some embodiments, the PTM can be charged or non-charged. Examples of non-charged PTM can include methylation. In some embodiments, the PTM can be negatively charged or positively charged. Exemplary negatively changed PTM include, but are not limited to, phosphorylation glycosylation and sialylation. In some embodiments, the PTM can be hydrophilic and/or hydrophobic.

In some embodiments, a library is generated comprising candidate pan-PTM binding antibodies by randomizing one or more regions outside the PTM binding pocket that binds to a context sequence adjacent to the PTM site. The context sequence adjacent to the PTM site can be of varying lengths. In some embodiments, the context sequence can be about 1-15 (i.e. 1, 2, 3, 4, 5, 6, 7, 8, 9, 10, 11, 12, 13, 14, or 15) amino acid residues upstream and/or downstream of the PTM site. In some embodiments, the context sequence can be about 15-30 (i.e. 15, 16, 17, 18, 19, 20, 21, 22, 23, 24, 25, 26, 27, 28, 29, or 30) amino acid residues upstream and/or downstream of the PTM site. In some embodiments, the context sequence can be both upstream and downstream of the PTM site. In some embodiments, the context sequence is located upstream of the PTM site. In some embodiments, the context sequence is located downstream of the PTM site.

Any method of site-directed mutagenesis known in the art can be used to randomize the one or more regions outside the PTM binding pocket that binds to the context sequence adjacent to the PTM site. For example, error-prone PCR, site directed mutagenesis by primer extension, inverse PCR, kunkel-based mutagenesis, cassette mutagenesis, whole plasmid mutagenesis, AXM mutagenesis, error-prone rolling circle amplification (RCA), or in vivo site-directed mutagenesis can be used to introduce randomization to generate the library. In some embodiments, kunkel-based site directed mutagenesis is used. In some embodiments, AXM mutagenesis is used. In some embodiments, error-prone rolling circle amplification (RCA) is used.

In some embodiments, the library is screened against the peptide or protein of interest with a PTM. In some embodiments, the library is screened against the peptide or protein of interest without a PTM. In some embodiments, this screen allows for the identification antibodies that specifically recognize the PTM. Alternatively, this screen allows for the identification of antibodies that do not recognize the PTM. Alternatively, this screen allows for the identification of antibodies that bind to epitopes that have and do not have the PTM. These latter antibodies are “pan-PTM” antibodies, as they bind to a specific epitope regardless of the PTM status of the epitope.

### Generation of Non-PTM-Binding Antibody Library

In some embodiments, a method is provided for the generation of non-PTM-binding antibodies that specifically bind a site without post translational modification (PTM) comprising: providing an antibody that specifically recognizes a PTM on a peptide or protein of interest; identifying a PTM binding pocket of the antibody; introducing an amino acid that repels the PTM at one or more sites in the PTM binding pocket of the antibody that are determined to interact with the PTM; generating a library comprising candidate non-PTM-binding antibodies by randomizing one or more regions outside the PTM binding pocket that bind to a context sequence adjacent to the PTM site; and screening the library against the peptide or protein of interest without PTM, thereby identifying non-PTM binding antibodies.

In some embodiments, a method of generating antibodies includes providing an antibody that specifically recognizes a PTM on a peptide or protein of interest. The PTM can be any PTM. Exemplary PTM modifications are described above. Having a starting antibody that recognizes a specific post-translational modification allows for an analysis and identification of residues that directly interact with the post-translational modification. In some embodiments, the starting antibody is used for structural analysis and for a determination of the antibody PTM binding pocket. In some embodiments, a PTM binding pocket of the starting antibody is identified. There are several methods in the art that allow for the identification of PTM binding pockets. Any suitable method can be used to identify the antibody PTM binding pocket. In some embodiments, one or more sites within the PTM binding pocket are structurally predicted. In some embodiments, one or more sites within the PTM binding pocket are experimentally determined.

In some embodiments, an amino acid that repels the PTM is introduced at one or more sites in the PTM binding pocket of the antibody that are determined to interact with the PTM. In embodiments, the amino acid that repels the PTM can be any amino acid. In embodiments, the amino acid that repels the PTM is positively charged or negatively charged. In embodiments, the amino acid that repels the PTM is negatively charged. In embodiments, the amino acid that repels the PTM is positively charged. In some embodiments, the amino acid that repels the PTM is positively charged or negatively charged depending on whether the charged amino can repel the PTM. In some embodiments, the amino acid that repels the PTM is negatively charged. For example, in some embodiments, the negatively-charged amino acid is phosphoserine (SEP), phophotyrosine, aspartic acid or glutamic acid. In embodiments, the negatively-charged amino acid is phosphoserine (SEP),In some embodiments, the amino acid that repels the PTM is positively charged. For example the positively charged amino acid can be lysine, arginine, or histidine.

The amino acid that repels the PTM can be a non-canonical amino acid. As non-limiting examples, the non-canonical amino acid is phosphoserine (SEP), phosphotyrosine, p-azido-phenylalanine, benzoyl-phenylalanine, or acetyl-lysine. As non-limiting examples, the amino acid can be selenocysteine or pyrrolysine. In embodiments, the amino acid that repels the PTM is introduced by a suppressor tRNA.

In some embodiments, the amino acid that repels the PTM is hydrophilic. In embodiments, any hydrophilic amino acid can be used. For example, the hydrophilic amino acid can be arginine, asparagine, aspartate, glutamine, glutamate, histidine, lysine, serine, or threonine.

In some embodiments, the amino acid that repels the PTM is hydrophobic. In embodiments, any hydrophobic amino acid can be used. For example, the hydrophobic amino acid is glycine, alanine, valine, leucine, isoleucine, proline, phenylalanine, methionine or tryptophan.

In some embodiments, a library is generated comprising candidate non-PTM binding antibodies by randomizing one or more regions outside the PTM binding pocket that bind to a context sequence adjacent to the PTM site. The context sequence adjacent to the PTM site can be of varying lengths. In some embodiments, the context sequence can be about 1-15 (i.e. 1, 2, 3, 4, 5, 6, 7, 8, 9, 10, 11, 12, 13, 14, or 15) amino acid amino acid residues upstream and/or downstream of the PTM site. In some embodiments, the context sequence can be about 15-30 (i.e. 15, 16, 17, 18, 19, 20, 21, 22, 23, 24, 25, 26, 27, 28, 29, or 30) amino acid residues upstream and/or downstream of the PTM site. In embodiments, the context sequence is located upstream of the PTM site. In embodiments, the context sequence is located downstream of the PTM site. In some embodiments, the context sequence can be both upstream and downstream of the PTM site.

Any method of site-directed mutagenesis known in the art can be used to randomize the one or more regions outside the PTM binding pocket that binds to the context sequence adjacent to the PTM site. For example, error-prone PCR, site directed mutagenesis by primer extension, inverse PCR, kunkel-based mutagenesis, cassette mutagenesis, whole plasmid mutagenesis, AXM mutagenesis, error-prone rolling circle amplificiation (RCA), or in vivo site-directed mutagenesis can be used to introduce randomization to generate the library. In some embodiments, kunkel-based site directed mutagenesis is used. In some embodiments, AXM mutagenesis is used. AXM mutagenesis is described in U.S. Pat. No. 9,617,537, and U.S. Pat. No. 9,422,549, the contents of each of which are incorporated herein by reference in their entireties. In some embodiments, error-prone rolling circle amplificiation (RCA) is used.

In some embodiments, the one or more regions outside the PTM binding pocket that bind to the context sequence are randomized without altering the PTM binding pocket.

In some embodiments, the library is screened against the peptide or protein of interest without a PTM. In some embodiments, this screen allows for the identification of antibodies that do not recognize the PTM.

### Generation of Inhibitor Antibodies

In embodiments, a method is provided for the generation of an inhibitor antibody. The inhibitor antibody can inhibit a peptide, or protein of interest by any method known in the art. For example, the inhibitor antibody produced by the methods herein can inhibit by steric inhibition, allosteric inhibition and/or by competitive inhibition. In embodiments, the antibody is a steric inhibitor. In embodiments, the antibody is an allosteric inhibitor. In embodiments, the antibody is a competitive inhibitor. In embodiments, the peptide or protein of interest is an enzyme. In embodiments, the peptide or protein of interest is a membrane-spanning protein. In embodiments, the peptide or protein of interest is a chimeric antigen receptor T-cell (CAR-T). In embodiments, the peptide or protein of interest is a cell receptor. The cell receptor can be any kind of cell receptor, for example, in embodiments the cell receptor is a G protein coupled receptor (GPCR), enzyme-coupled receptor, or a ligand-gated ion channel receptor. The method of producing inhibitor antibodies that target a peptide or protein of interest can be applied to any kind of GPCR. Numerous kinds of G protein coupled receptors are known in the art. For example, GPCRs are found as adenosine receptors, adhesion GPCRs, adrenergic receptors, chemokine receptors, cholecystokinin receptors, dopamine receptors, histamine receptors, metabotropic glutamate receptors, muscarinic acetylcholine receptors, olfactory receptors, opioid receptors and serotonin receptors, among others. In embodiments, the peptide or protein of interest is selected from Leptin Receptor, Glucagon Receptor, Insulin Receptor, CXCR4, NTSR1, NTSR2 and Receptor Tyrosine Kinase.

In embodiments, the inhibitor antibody is an anti-idiotype antibody. The inhibitor antibody can be constructed to target any kind of idiotype. In embodiments, the inhibitor antibody inhibits Herceptin, and/or anti-CD19. In embodiments, the inhibitor antibody inhibits a bispecific antibody. In embodiments, the inhibitor antibody binds to both denatured and native IgG. In embodiments, the inhibitor antibody binds to either denatured or native IgG.

In embodiments, the method of generating inhibitor antibodies to the peptide or protein of interest includes providing a modified peptide of interest modified to include a negatively charged amino acid, screening the modified peptide against an antibody library biased towards the negatively charged amino acid, isolating one or more antibodies that bind to the modified peptide of interest; and determining the binding of the one or more antibodies to the peptide of interest without the modification, thereby identifying an antibody that binds to the peptide of interest.

In embodiments, the method of producing an antibody to a peptide of interest, includes: providing a modified peptide of interest modified to include a negatively charged amino acid, screening the modified peptide against an antibody library biased towards the negatively charged amino acid, isolating one or more antibodies that bind to the modified peptide of interest; generating a library of clonotypes of the one or more antibodies by affinity maturation; screening the library of the clonotypes against the peptide of interest without the modification, thereby identifying an antibody that binds to the peptide of interest.

In embodiments, the peptide that forms the base sequence for the modified peptide corresponds to an ectodomain and/or surrounding regions of the ectodomain of a peptide or protein of interest. In embodiments, the peptide that forms the base sequence for the modified peptide is the ectodomain of the peptide or protein of interest. In embodiments, the peptide that forms the base sequence for the modified peptide is the active site and/or surrounding regions of the active site obtained from the protein of interest. In embodiments, the peptide or protein of interest is an enzyme. By “surrounding regions” is meant 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids upstream and/or downstream from the ectodomain and/or active site. In embodiments, the modified peptide is produced by introducing one or more amino acids (e.g. 1, 2, 3, 4, 5, 6, 7, 8, 9, or 10) into a base sequence obtained from the peptide or protein of interest as described above. In embodiments, the modified peptide is produced by introducing one amino acid into a base sequence obtained from the peptide or protein of interest. By “introducing” is meant any one of the following: i) replacing an existing amino acid in its native position with another, selected amino acid; ii) adding an additional, selected amino acid into a sequence at a selected position; or iii) removing a selected amino acid from a sequence and adding another amino acid to a distinct position in the sequence apart from the position of the original, removed amino acid. In embodiments, the modified peptides are synthesized to include negatively-charged amino acid. In embodiments, anti-idiotype antibodies are constructed through use of a base sequence from CDRs H3 and/or L3 to create a modified peptide. In embodiments, the CDR H3 sequence is used as a base sequence to synthesize a modified peptide. In embodiments, the CDR L3 is used as a base sequence to synthesize a modified peptide. In embodiments, both CDR H3 and L3 are used as base sequences to synthesize modified peptides.

In some embodiments, the negatively-charged amino acid is a phosphorylated amino acid. In some embodiments, the negatively-charged amino acid is phosphoserine (SEP), phosphotyrosine, aspartic acid or glutamic acid. In embodiments, the negatively-charged amino acid is phosphoserine (SEP). The isolectric point (pI) of an amino acid can be used as a guide in selecting the negatively-charged amino acid (Table 1 above). The negatively-charged amino acid is introduced into the base peptide, thus forming the modified peptide. About 1, 2, 3, 4, 5, 6, 7, 8, 9, 10, 11, 12, 13, 14, or 15 amino acids can be introduced within the base peptide. In embodiments, the negatively-charged amino acid is introduced to 1 site within the base peptide to produce the modified peptide. In embodiments, the negatively-charged amino acid is introduced adjacent to the ectodomain or active site. By “adjacent” is meant about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids upstream or downstream from the ectodomain and/or active site.

Any method in the art can be used to screen for candidate antibodies. In some embodiments, an emulsion whole cell based library screening method is used. In embodiments, the emulsion whole cell based library screening method is delayed emulsion infectivity (“DEI”). Whole cell screening methods (e.g. whole cell panning) are described in US 2015-0322150 and in WO/2015/085079, the contents of each of which are hereby incorporated by reference in their entireties.

Any method known in the art can be used to purify the inhibitor antibodies. Numerous antibody purification techniques are available in the art. For example, antibodies can be purified by affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography.

In embodiments, assays are used to test for the ability of the purified inhibitor antibody to bind and/or inhibit the peptide or protein of interest. In embodiments, cell-based assays are used to test the binding and/or inhibiting ability of the purified inhibitor antibody. Other assays can be used to test the purified inhibitor antibody, for example, ELISA, FACS, and/or immunocytochemistry.

Any antibody library can be used with the methods disclosed herein. In embodiments, the antibody library is a negative charge biased antibody library. In embodiments, the antibody library is a phopho-biased antibody library.

In embodiments, a library of clonotypes of one or more antibodies are mutated and matured by means known in the art. In embodiments, the library of clonotypes of one or more antibodies are mutated and matured by affinity maturation. In embodiments, the library of clonotypes of one or more antibodies are matured by AXM Mutagenesis (FIG. 7).

In embodiments, the antibody library that is screened against the modified peptide has a pre-defined propensity to bind a phosphorylated amino acid (e.g. phosphoserine (SEP) or phosphotryosine). In embodiments, the antibody library screened against the modified peptide has a general mode to bind specific sequences in the context of the modified sequence (i.e. the context sequence). In embodiments, the phage library is constructed and used to identify anti-phospho-peptide antibodies. In embodiments, the phosphorylated amino acid introduced into the peptide being screened replaces the natural amino acid at that sequence. In embodiments, a directed evolution approach of the isolated antibodies is performed. Following directed evolution, the antibodies are screened for clonotypes that are able to recognize the native sequence (e.g. where the naturally occurring amino acid in the peptide being screened replaces the phosphoryolated amino acid at that sequence).

In embodiments, the base sequence for the modified epitope is selected by systematically “walking” down approximately every 1^{st}, 2^{nd}, 3^{rd}, 4^{th}, 5^{th}, 6^{th}, 7^{th}, 8^{th}, 9^{th}, 10^{th}, 11^{th}, or 12^{th} amino acid in or adjacent to the external domain or active site of the peptide or protein of interest. In embodiments, the base sequence for the modified epitope is selected by systematically “walking” down approximately every 1^{st}, 2^{nd}, 3^{rd}, 4^{th}, 5^{th}, 6^{th}, 7^{th}, 8^{th}, 9^{th}, 10^{th}, 11^{th}, or 12^{th} amino acid in the external domain or active site of the peptide or protein of interest. In embodiments, the base sequence for the modified epitope is selected by systematically “walking” down approximately every 1^{st}, 2^{nd}, 3^{rd}, 4^{th}, 5^{th}, 6^{th}, 7^{th}, 8^{th}, 9^{th}, 10^{th}, 11^{th}, or 12^{th} amino acid in the external domain of the peptide or protein of interest. In embodiments, the base sequence for the modified epitope is selected by systematically “walking” down approximately every 1^{st}, 2^{nd}, 3^{rd}, 4^{th}, 5^{th}, 6^{th}, 7^{th}, 8^{th}, 9^{th}, 10^{th}, 11^{th}, or 12^{th} amino acid in the active site of the peptide or protein of interest. In embodiments, the base sequence for the modified epitope is selected by systematically “walking” down approximately every 1^{st}, 2^{nd}, 3^{rd}, 4^{th}, 5^{th}, 6^{th}, 7^{th}, 8^{th}, 9^{th}, 10^{th}, 11^{th}, or 12^{th} amino acid in or adjacent to the external domain or in or adjacent to the active site of the peptide or protein of interest.

### Library Construction, Amplification and Screen

The antibody library can be any kind of antibody library. Examples of antibody libraries include phage display, bacterial display, yeast display, ribosome display and mRNA display. In some embodiments, the antibody library is phage display.

Any kind of library amplification methods known in the art can be used for amplification of the second library. In some embodiments, a sequence of interest may be amplified using a pair of oligonucleotides, of which one oligonucleotide is a protected oligonucleotide and the other is a non-protected oligonucleotide. The sequence of interest may be amplified using such an oligonucleotide pair by an amplification reaction such as PCR, error-prone PCR, isothermal amplification, or rolling circle amplification. In some embodiments, the library amplification method is rolling circle amplification (RCA). In some embodiments, the library amplification method is error-prone rolling circle amplification. RCA can amplify the library by about between 50- and 150-fold (e.g. 50, 55, 60, 65, 70, 75, 80, 85, 90, 95, 100, 105, 110, 115, 120, 125, 130, 135, 140, 145, 150, and any values in between). In some embodiments, RCA can amplify the library by about 100-fold. In some embodiments, the RCA amplified library is linearized and re-circularized.

The antibody library can be introduced into any suitable cell known in the art. In embodiments, the cell is an archaeal cell, prokaryotic cell, bacterial cell, fungal cell, or eukaryotic cell. In embodiments, the cell is a yeast cell, plant cell, or animal cell. In embodiments, the cell is an E. coli cell or S. cerevisiae cell. In some embodiments, the cell is an insect cell. In embodiments, the cell strain is any electro- or chemically competent cell. In some embodiments, the library is transformed into DH5a, JM109, C600, HB101, or TG1. In some embodiments, the library is transformed into TG1 cells.

In some embodiments, the antibody library has a diversity of about between 10⁷ and 10¹⁴ (i.e. 10⁷, 10⁸, 10⁹, 10¹⁰, 10¹¹, 10¹², 10¹³, and 10¹⁴) unique antibodies. In some embodiments, the library has a diversity of at least 10⁸ and 10¹² (i.e. 10⁸, 10⁹, 10¹⁰, 10¹¹, and 10¹²).

Any method in the art can be used to screen for candidate antibodies. In some embodiments, an emulsion whole cell based library screening method is used. Whole cell screening methods (e.g. whole cell panning) are described in US 2015-0322150 and in WO/2015/085079, the contents of each of which are hereby incorporated by reference in their entireties. Whole cell screening methods include, for example, the creation of an emulsion in which E. coli that have been transduced with the antibody phage library are incubated with cells or beads that display the antigen of interest. During an overnight incubation process, antibody-displaying phages are secreted from the E. coli and attach to the antigen presenting cells or beads. Subsequent processing includes the addition of labeled antibodies that attach to the phage, and subsequent FACS sorting to isolate the antibody-displaying phage that have bound to the antigens displayed on the whole cell or beads. In some embodiments, the library is processed for multiple rounds of whole cell screening. In some embodiments, whole cell screening is performed between about 3 to 8 times (i.e. 3, 4, 5, 6, 7, 8). In some embodiments, whole cell screening is performed about 3 times. In some embodiments, multiple rounds of whole cell screening results in the isolation of more specific epitope-binding antibodies.

### Delayed Emulsion Infectivity (DEI): Recombinatorial Biopanning

In embodiments, the whole cell panning approach includes the following: i) providing a bacterial cell (e.g. E. coli) that has an inducible phage attachment site and that displays on the surface a peptide, protein, phosphorylated peptide or phosphorylated protein; ii) the bacterial cells are mixed, emulsified with a phage antibody library comprising a bar-code sequence identifier at a ratio such that there is one phage per one bacterial cell; iii) inducing expression of the inducible phage attachment site, thus allowing for phage infection of the bacterial cell; iv) growing the phage-infected bacterial cell, and v) sequencing the bar-code sequence identifiers to identify the antibody encoded by the phage. In embodiments, the bacterial cell is an E. coli cell.

As background, the F-pilus is the attachment site for M13 infection of E. coli. The F-pilus is not expressed in F⁺ E. coli grown at <22° C. In DEI, phagemid-encoded antibody ‘association’ to the cells relies on the interaction of an M13-displayed antibody and the bacterial cell surface-displayed antigen of a genotypic F⁺ E. coli host that has been grown overnight at 16° C. These phenotypic F⁻ hosts are mixed at a 10:1 (cells:phage) ratio to a M13 phage Ab library. A desired ratio is to have 1 phage associated per bacterial cell. By having a ratio consisting of more bacteria than phage, limitations are overcome due to Poisson distribution. The antibody library and cell library mix is washed to remove unattached- and weakly-attached phage and resuspended in growth medium. Single washed cells along with any attached phage are emulsified in a growth medium-in-oil emulsion. By producing more, and smaller microdroplets than bacteria, overcoming Poisson distribution. The phospho-focusing of the library due to three amino acids in CDR H2 increases its effective size approximately 8,000 fold (=20³) and compensates for using smaller libraries in DEI. The emulsion temperature is increased to 37° C. to: (1) induce F pilus expression, (2) allow phage disassociation from the cell surface and (3) enable disassociated phage infection via F-pilus attachment. Within the cell, the incoming antibody-encoded donor phagemid is recombined with an antigen-encoded acceptor plasmid through the mediation of phiC31 integrase. A chloramphenicol (CAM) gene is turned on because of the successful recombination of the donor phagemid and acceptor plasmid to form the plasmid co-integrant. After several hours, the emulsion is broken and the cells grown overnight in the presence of CAM. The recombined plasmids from the pool, which now have the Ab gene physically linked to the Ag sequence is isolated. Paired-end Next Generation DNA sequencing (NGS) of antigen and antibody sequences of individual plasmids and a multiplex barcoding scheme is used to identify and link all of the CAM-resistant antibody:antigen pairs.

In embodiments, the E. coli cell is an F⁺ E. coli cell. In embodiments, the F⁺ E. coli cell contains an acceptor plasmid comprising a chloramphenicol (CAM) gene. In embodiments, the F⁺ E. coli cell is grown at a temperature of less than about 22° C. (e.g. about 10.0° C., 10.5° C., 11.0° C., 11.5° C., 12.5° C., 13.0° C., 13.5° C., 14.0° C., 14.5° C., 15.0° C., 15.5° C., 16.0° C., 16.5° C., 17.0° C., 17.5° C., 18.0° C., 18.5° C., 19.0° C., 19.5° C., 20.0° C., 20.5° C., 21.0° C., 20.5° C.). In embodiments, the F⁺ E. coli are mixed with the phage library, thus emulsifying the F⁺ E. coli cell. In embodiments, the emulsified F⁺ E. coli cell are grown at a temperature of about 37° C., followed by introducing chloramphenicol into the culture. In embodiments, plasmids contained within the infected, grown, chloramphenicol-exposed F⁺ E. coli are sequenced to identify the antibody encoded by the phage.

In some embodiments, the method further comprises a validating step. Any method known in the art can be used in the validating step in order to validate the antibodies of interest. For example, ELISA and/or functional assays can be used for antibody validation. In some embodiments, the validating step is high throughput.

In some embodiments, the validating includes determining whether the identified PTM, pan-PTM, non-PTM binding antibodies are steric inhibitors against PTM. For example, the methods provided herein can be used to generate steric inhibitory antibodies that target a peptide or protein of interest. In embodiments, the methods described herein generate steric inhibitory antibodies that inhibit an enzyme of interest. The method for generating steric inhibitory antibodies includes, for example, providing an antibody that specifically recognizes a PTM on an enzyme of interest; identifying a PTM binding pocket of the antibody; generating a library comprising candidate pan-PTM binding antibodies by randomizing one or more regions inside or outside the PTM binding pocket that bind to the PTM site on the enzyme; screening the library against the enzyme of interest and selecting the antibodies that recognize the PTM site in the absence of PTM modification; and selecting steric inhibitory antibodies by performing an enzymatic activity assay. For these methods, any enzyme can be the target for steric antibody inhibitors. Assays to detect or quantify enzymatic activity are known in the art. Any such enzymatic activity assay can be used in the selecting process.

### Phospho-Status Specific Antibody Library Design

In some embodiments, a method is provided for the generation of phosphorylation specific antibody libraries. In some embodiments, a method is provided for the generation of pan-phosphorylation antibody libraries. In some embodiments, a method is provided for the generation of non-phosphorylation antibody libraries.

The following is an exemplary strategy for creating antibody libraries that are specific for phosphorylation post-translational modification.

In some embodiments, the phosphorylation post-translational modification (PTM) can be at any residue. In some embodiments, the phosphorylation occurs on serine, threonine, tyrosine and/or histidine residues. In some embodiments, the phosphorylation occurs on serine, threonine, tyrosine, histidine, arginine and/or lysine residues. In some embodiments, the PTM site is pSer. In some embodiments, the PTM site is pTyr. In some embodiments, the PTM site is pThr. In some embodiments, the PTM site is pHis. In some embodiments, the PTM site is pArg. In some embodiments, the PTM site is pLys.

In some embodiments, a method of generating antibodies includes providing an antibody that specifically recognizes a PTM on a peptide or protein of interest. In some embodiments, the PTM is phosphorylation. In some embodiments, the phosphorylation binding pocket is identified.

Previous studies have suggested that the ability of an antibody to bind phosphorylated amino acids is dependent on the heavy/light chain complementarity-determining region 2 (CDR H2 or L2), which makes direct contacts with the phosphorylated-amino acid. Further studies have shown that phospho-focused libraries with a propensity to bind phosphorylated peptides could be produced with a natural phosphate-binding motif in CDR H2.

Through random, independent screening, amino acids in CDR H2 of an anti-Myb pSer specific scFv were shown to be critical for binding the phosphorylated Myb₁₋₂₀ peptide. The choice of L2 or H2 appears to correlate with the direction of the context sequence. Based on these observations, phosphate binding sites can be designed as a localized module.

Structural analysis of phosphorylation-antibody binding interactions indicated that with regard to phosphorylated epitopes, the phosphorylated regions usually bare linear motifs with the structure of loops or turns. Without wishing to be bound by theory, this is presumably due to the need to fit into the narrow substrate-binding pocket of kinases. As such, sequence variation among different phosphorylated regions actually does not affect the relatively uniform secondary structure.

Further analyses suggest that phospho-epitope-specificity is likely to require an optimal length for a context sequence. Phospho-specific Abs recognize both the phosphorylated amino acid and the surrounding context sequence. The successful phosphorylated peptide Abs usually recognize about 4-5 extra residues upstream and/or downstream of the phosphorylated residue, suggesting excess binding to context sequence might dilute the contribution of the phosphorylated amino acid and consequently impair phospho-peptide specificity over the native peptide.

In some embodiments, the optimal length of a context sequence is about 1, 2, 3, 4, 5, 6, 7, 8, 9, 10, 11, 12, 13, 14, or 15 residues upstream or downstream of the phosphorylated residue. In some embodiments, the optimal length of a context sequence is about 3, 4, 5, or 6 extra residues upstream or downstream of the phosphorylated residue. In some embodiments, the optimal length of a context sequence is about 4 or 5 extra residues upstream or downstream of the phosphorylated residue. In some embodiments, the context sequence can be upstream or downstream of the post-translational modification. In some embodiments, the context sequence is upstream of the post-translational modification. In some embodiments, the context sequence is downstream of the post-translational modification.

Collectively, the data indicate that the uniform structure, the likely defined context length, as well as the proven design of phosphate binding sites altogether make a highly focused, knowledge based phospho-status binding library designable if a common binding mode of context sequence could be explored.

In some embodiments, the binding pocket for phosphorylation post-translational modification is CDR H2 region and/or a CDR L2 region. In some embodiments, regions outside of the CDR H2 region and/or CDR L2 region are randomized. Any region outside of the CDR H2 region and/or CDR L2 region can be randomized. For example, CDR H1, H3, L1, L3, or the framework regions can be randomized. In some embodiments, one or more randomized regions comprise randomized CDR H3, or L3. In some embodiments, the PTM binding pocket comprises a CDR H2 region, and the one or more randomized regions comprise randomized CDR H3, CDR L3, and/or CDR L2

Any method of site-directed mutagenesis known in the art can be used to randomize the one or more regions outside the phosphorylation binding pocket that binds to the context sequence adjacent to the phosphorylation site. For example, error-prone PCR, site directed mutagenesis by primer extension, inverse PCR, kunkel-based mutagenesis, cassette mutagenesis, whole plasmid mutagenesis, AXM mutagenesis, error-prone rolling circle amplificiation (RCA), or in vivo site-directed mutagenesis can be used to introduce randomization to generate the library. In some embodiments, kunkel-based site directed mutagenesis is used. In some embodiments, AXM mutagenesis is used. In some embodiments, error-prone rolling circle amplificiation (RCA) is used.

In some embodiments, the one or more regions outside the phosphorylation binding pocket that bind to the context sequence are randomized without altering the phosphorylation binding pocket.

In some embodiments, the library is screened against the peptide or protein of interest with a phosphorylation. In some embodiments, the library is screened against the peptide or protein of interest without a PTM. In some embodiments, this screen allows for the identification antibodies that specifically recognize the phosphorylation. Alternatively, this screen allows for the identification of antibodies that do not recognize the phosphorylation. Alternatively, this screen allows for the identification of antibodies that bind to epitopes that have and do not have the phosphorylation. These latter antibodies are “pan-phosphorylation” antibodies, as they bind to a specific epitope regardless of the phosphorylation status of the epitope.

Using this approach, which is a combination of functional mutagenesis and structural modeling, along with a comparison with existing structures of specific post-translational modification antibody complexes, the candidate peptide binding groove can be identified. For the phospho-peptide antibody complexes, the peptide binding groove was identified as holstered by several restricted regions of CDR L3 and H3, which could serve as the candidate for a focused phospho-peptide library design.

### Antibodies

An antibody of the present disclosure may be multispecific, e.g., bispecific. An antibody of the may be mammalian (e.g., human or mouse), humanized, chimeric, recombinant, synthetically produced, or naturally isolated. Exemplary antibodies of the present disclosure include, without limitation, IgG (e.g., IgG1, IgG2, IgG3, and IgG4), IgM, IgA (e.g., IgA1, IgA2, and IgAsec), IgD, IgE, Fab, Fab′, Fab′2, F(ab′)2, Fd, Fv, Feb, scFv, scFv-Fc, and SMIP binding moieties. In certain embodiments, the antibody is an scFv. The scFv may include, for example, a flexible linker allowing the scFv to orient in different directions to enable antigen binding. In various embodiments, the antibody may be a cytosol-stable scFv or intrabody that retains its structure and function in the reducing environment inside a cell (see, e.g., Fisher and DeLisa, J. Mol. Biol. 385(1): 299-311, 2009; incorporated by reference herein). In particular embodiments, the scFv is converted to an IgG or a chimeric antigen receptor according to the methods described herein. In embodiments, the antibody binds to both denatured and native protein targets. In embodiments, the antibody binds to either denatured or native protein.

In most mammals, including humans, whole antibodies have at least two heavy (H) chains and two light (L) chains connected by disulfide bonds. Each heavy chain consists of a heavy chain variable region (VH) and a heavy chain constant region (CH). The heavy chain constant region consists of three domains (CH1, CH2, and CH3) and a hinge region between CH1 and CH2. Each light chain consists of a light chain variable region (VL) and a light chain constant region (CL). The light chain constant region consists of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

Antibodies of include all known forms of antibodies and other protein scaffolds with antibody-like properties. For example, the antibody can be a monoclonal antibody, a polyclonal antibody, human antibody, a humanized antibody, a bispecific antibody, a monovalent antibody, a chimeric antibody, or a protein scaffold with antibody-like properties, such as fibronectin or ankyrin repeats. The antibody can have any of the following isotypes: IgG (e.g., IgG1, IgG2, IgG3, and IgG4), IgM, IgA (e.g., IgA1, IgA2, and IgAsec), IgD, or IgE.

An antibody fragment may include one or more segments derived from an antibody. A segment derived from an antibody may retain the ability to specifically bind to a particular antigen. An antibody fragment may be, e.g., a Fab, Fab′, Fab′2, F(ab′)2, Fd, Fv, Feb, scFv, or SMIP. An antibody fragment may be, e.g., a diabody, triabody, affibody, nanobody, aptamer, domain antibody, linear antibody, single-chain antibody, or any of a variety of multispecific antibodies that may be formed from antibody fragments.

Examples of antibody fragments include: (i) a Fab fragment: a monovalent fragment consisting of VL, VH, CL, and CH1 domains; (ii) a F(ab′)2 fragment: a bivalent fragment comprising two Fab fragments linked by a disulfide bridge at the hinge region; (iii) a Fd fragment: a fragment consisting of VH and CH1 domains; (iv) an Fv fragment: a fragment consisting of the VL and VH domains of a single arm of an antibody; (v) a dAb fragment: a fragment including VH and VL domains; (vi) a dAb fragment: a fragment that is a VH domain; (vii) a dAb fragment: a fragment that is a VL domain; (viii) an isolated complementarity determining region (CDR); and (ix) a combination of two or more isolated CDRs which may optionally be joined by one or more synthetic linkers. Furthermore, although the two domains of the Fv fragment, VL and VH, are coded for by separate genes, they can be joined, using recombinant methods, e.g., by a synthetic linker that enables them to be expressed as a single protein, of which the VL and VH regions pair to form a monovalent binding moiety (known as a single chain Fv (scFv)). Antibody fragments may be obtained using conventional techniques known to those of skill in the art, and may, in some instances, be used in the same manner as intact antibodies. Antigen-binding fragments may be produced by recombinant DNA techniques or by enzymatic or chemical cleavage of intact immunoglobulins. An antibody fragment may further include any of the antibody fragments described above with the addition of additional C-terminal amino acids, N-terminal amino acids, or amino acids separating individual fragments.

An antibody may be referred to as chimeric if it includes one or more antigen-determining regions or constant regions derived from a first species and one or more antigen-determining regions or constant regions derived from a second species. Chimeric antibodies may be constructed, e.g., by genetic engineering. A chimeric antibody may include immunoglobulin gene segments belonging to different species (e.g., from a mouse and a human).

An antibody may be a human antibody. A human antibody refers to a binding moiety having variable regions in which both the framework and CDR regions are derived from human immunoglobulin sequences. Furthermore, if the antibody contains a constant region, the constant region also is derived from a human immunoglobulin sequence. A human antibody may include amino acid residues not identified in a human immunoglobulin sequence, such as one or more sequence variations, e.g., mutations. A variation or additional amino acid may be introduced, e.g., by human manipulation. A human antibody of the present disclosure is not chimeric.

An antibody may be humanized, meaning that an antibody that includes one or more antigen-determining regions (e.g., at least one CDR) substantially derived from a non-human immunoglobulin or antibody is manipulated to include at least one immunoglobulin domain substantially derived from a human immunoglobulin or antibody. An antibody may be humanized using the conversion methods described herein, for example, by inserting antigen-recognition sequences from a non-human antibody encoded by a first vector into a human framework encoded by a second vector. For example, the first vector may include a polynucleotide encoding the non-human antibody (or a fragment thereof) and a site-specific recombination motif, while the second vector may include a polynucleotide encoding a human framework and a site-specific recombination complementary to a site-specific recombination motif on the first vector. The site-specific recombination motifs may be positioned on each vector such that a recombination event results in the insertion of one or more antigen-determining regions from the non-human antibody into the human framework, thereby forming a polynucleotide encoding a humanized antibody.

In certain embodiments, an antibody is converted from scFv to an IgG (e.g., IgG1, IgG2, IgG3, and IgG4). There are various methods in the art for converting scFv fragments to IgG. One such method of converting scFv fragments to IgG is disclosed in US patent application publication number 20160362476, the contents of which are incorporated herein by reference.

### Binding Affinity

Binding affinity for antibodies and antibody fragments can be determined through various methods known in the art. For example, binding affinity can be determined by the Scatchard analysis of Munson and Pollard, Anal. Biochem., 107:220 (1980). Another method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473).

In some embodiments, the post-translational specific antibody binding affinity is about a K_{d} of 1 nM. In some embodiments, the post-translational specific antibody binding affinity is greater than about a K_{d} of 1 nM. In some embodiments, the antibody binding affinity is about between a K_{d} of 1 and 50 nM (i.e. 1, 2, 3, 4, 5, 6, 7, 8, 9, 10, 15, 20, 25, 30, 35, 40, 45, 50 nM, or any values in between). In some embodiments, the antibody binding affinity is about between a K_{d} of 1 and 15 nM (i.e. 1, 2, 3, 4, 5, 6, 7, 8, 9, 10, 11, 12, 13, 14, 15 or any values in between).

### EXAMPLES

### Example 1

### Identification of Antibody Peptide Binding Groove for Specific Post-Translational Modification

This example describes the identification of an antibody peptide binding groove that binds a specific post-translational modification. The specific post-translational modification assessed in this example is phosphorylation. A schematic that shows a method of one embodiment that illustrates the derivation of post-translational modification-specific antibody clonotypes is shown in FIG. 1.

Using a combination of functional mutagenesis and structural modeling as well as a comparison with existing structures of phospho-peptide Ab complexes, a shared peptide binding groove was identified that is holstered by several restricted regions of CDR L3 and H3. These restricted regions in CDR L3 and H3 can serve as a candidate for a focused phospho-peptide library design.

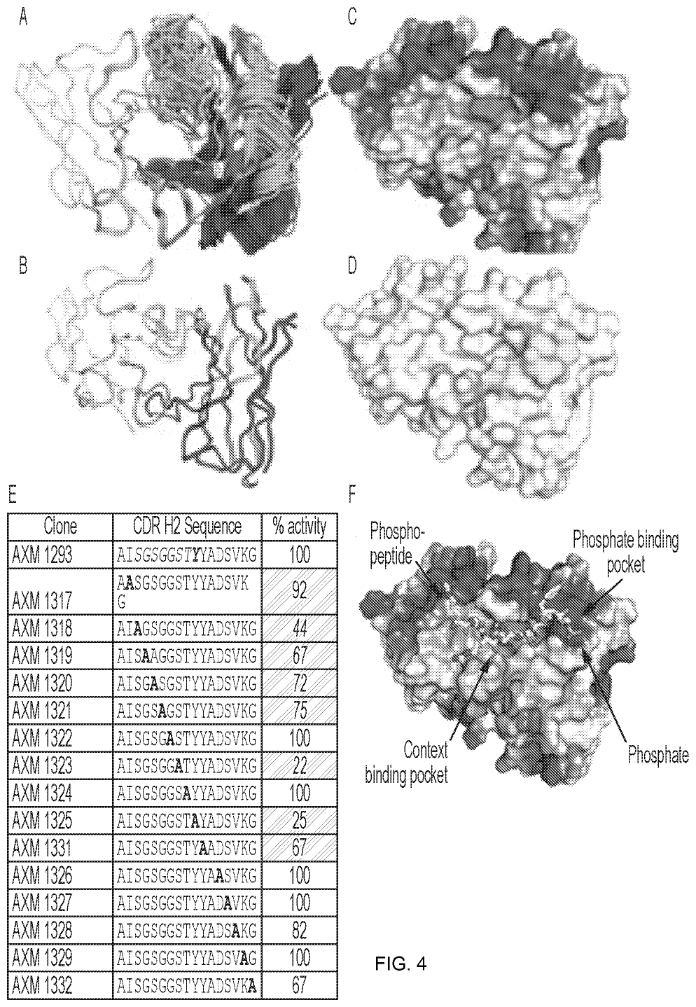

Data obtained from random alanine scanning mutagenesis of CDR regions of an anti-Myb phosphorylated serine (pSer)-specific scFv (AXM1293), showed that amino acid residues in CDR H2 were critical for the binding of the phosphorylated Myb₁₋₂₀ peptide (FIG. 4). Based on the alanine scanning results (FIG. 4) and the finding that H2 is the phosphate-binding center, the Ab structure of anti-Myb pSer specific scFv (AXM1293) and its complex with its cognate phosphorylated peptide was modeled using the RosettaAb3 software. From the analysis of results obtained from structural modeling and alanine scanning mutagenesis (FIG. 4), it was concluded that L2 and H2 can be utilized to target phospho-peptides with C-terminal or N-terminal context sequences, respectively (FIG. 4). It was also determined that two other phosphopeptide-Ab structures, although possessing diverse phosphate binding sites (H2 or L2), shared a common context-sequence binding mode, namely, that the peptide is holstered by H3 and L3, which is similarly observed in the complex model (FIG. 4). Further validations will be performed using mutagenesis and crystallographic methods.

### Example 2

### The Design and Production of Post-Translational Modification-Specific Antibody Libraries

This example describes the design and production of focused phosphorylated serine (pSer), phosphorylated tyrosine (pTyr), and pSer/pTyr antibody libraries. From an analysis of prior elucidated structures and the data generated herein, it was determined that the core binding region of the phosphopeptide can be localized to a triangular region including L3, H3 and H2 or L2. The major diversity of this interaction was contributed by L3 and H3, while the choice of H2 or L2 dictates the phosphate binding and possibly the peptide direction.

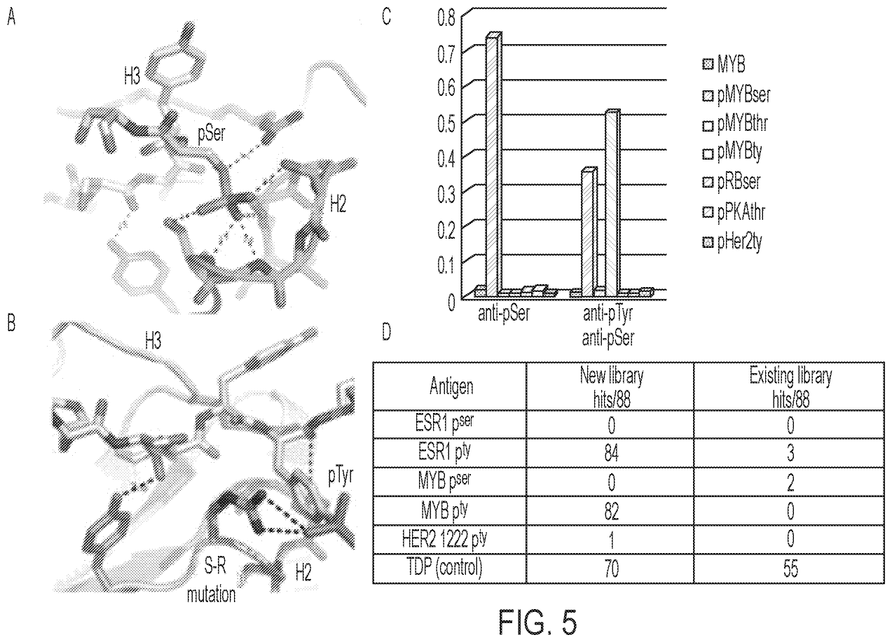

Antibodies will be selected that are pSer-specific, such as AXM1293, and additional antibody clones will be selected that are anti-pTyr specific, or both anti-pSer/p-Tyr specific (see FIG. 5). Parts of L3 and H3 will be randomized, that is, those CDR loops that are predicted to mediate the interaction with the specific peptide/protein will be randomized, while leaving CDR H2, which already has an affinity for pSer, pTyr or pSer/pTyr, unchanged. Thus, this will provide a simple switch to shift the pSer antibody library to pTyr recognition, and, more importantly, supports the computational model that the H2 region indeed contacts the peptide. To this end, a library against pTyr peptides has been obtained, where residues on L3, H3 and L2 were randomized. The data obtained from this library indicates a promising success rate against pTyr-peptides compared with a traditional library (FIG. 5).

To further confirm that the side chains of the context sequence mainly bind in-between L3 and H3, other interacting amino acids in CDRs L3 and H3 will be investigated by alanine scanning. Further confirmation of the computational models and to pinpoint residues that interact with a peptide will be conducted, including, for example, crystallizing the phosphorylated-peptide and Ab complex. Variant libraries will also be made by changing the charged amino acids surrounding the context groove to produce the variant libraries. This will allow for an expansion of peptides we can successfully screen.

### Antibody Library Design

For the AXM1293 (anti-Myb pSer11) library, 12 residues will be randomized in CDRs L3 (L93, L94, L95 and L95A in Chothia numbering), and H3 (H95, H96, H97, H98, H99, H100, H100A and H100B in Chothia numbering) based on the potential interacting (within 4 Å of the peptide) residues as well as their diversity in sequenced human Abs in the Kabat database. Notably, H95 (an Asp in AXM1293) contributes largely to the negative charge of the binding pocket, which complements the positive charge of the binding epitope. Sets of residues will be chosen to ensure charge complementarity against future targets. At least three- and up to six libraries with defined H2 regions targeting pSer or pTyr peptide will be constructed in parallel.

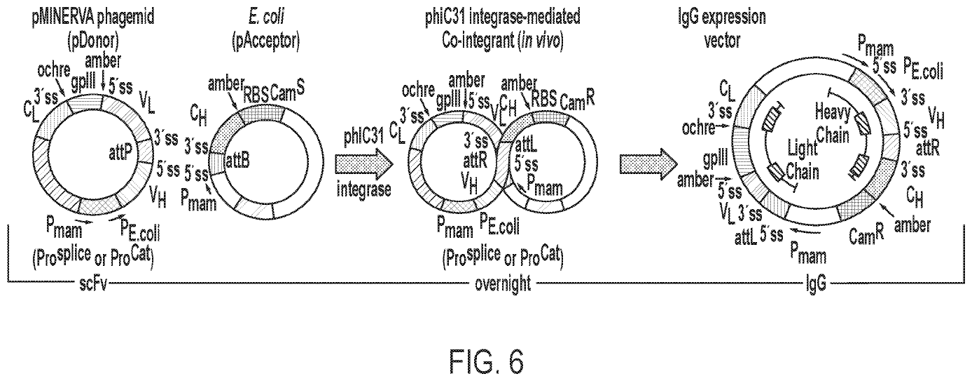

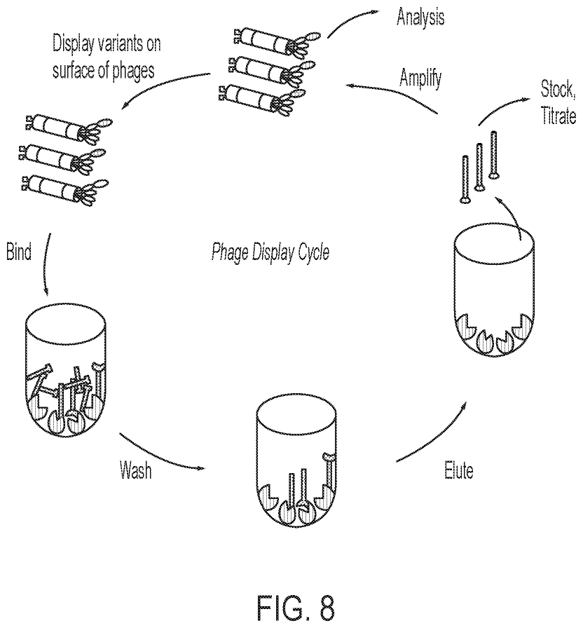

Libraries were constructed in a modified pMINERVA phagemid system (see FIG. 6). To construct the libraries, a modification of AXM mutagenesis was used (FIG. 5). The modification of AXM mutagenesis uses a Kunkel-based site directed mutagenesis of residues at a chosen CDR positions in the Kabat database. The library of scFvs will be displayed on the surface of bacteriophage M13 as a genetic fusion to the gpIII coat protein (FIG. 8). Since preliminary libraries were designed without the guidance of the structural model, a more focused library with randomization of only potential phospho-peptide contacting residues is likely to enhance the screening efficiency. Data from phage libraries demonstrated enhanced biopanning results when using a phospho-focused antibody library (See Table 2 and 3 below).

TABLE 2
A Phospho-Focused Antibody Library Results in
Enhanced Biopanning - Phospho-Serine Targets

| | Discovery Hits from a Naïve | | DisMat Hits from a PhosphoSerine- | |
|---|---|---|---|---|
| | Phospho-focused Library | | specific Discovery Hit | |
| | Cell-supernatant ELISA | | Cell-supernatant ELISA | |
| | PhosphoSerine- | PhosphoSerine- | Phospho- | Serine |
| TARGET | specific | preferred | Independent* | Specific* |
| p4_p_Smad2 | 87 | 11 | 72 (3 clones) | 0 |
| p5_p_CREB | 2 | 39 | 78 (1 clone) | 3 |
| p6_p_HtrA2 | 5 | 3 | 47 (1 clone) | 1 |
| p15_p_p70s6_444 | 8 | 0 | 20 (1 clone) | 0 |
| p11_p_akt1_473 | 15 | 4 | 30 (1 clone) | 35 |
| p3_p_RIPK3 | 3 | 5 | 34 (1 clone) | 0 |
| p10_p_Sgk1 | 29 | 23 | 132 (3 clones) | 0 |
| p2_p_RIPK3 | 140 | 4 | 0 (1 clone) | 0 |
| p14_p_p53_15 | 84 | 8 | 39 (1 clone) | 0 |
| P13_p_p70s6_389aka412 | 84 | 2 | 0 (1 clone) | 0 |
| Trim33_pS1119 | 8 | 0 | 5 (1 clone) | 0 |
| BRD2_pS37 | 0 | 0 | — | — |
| MTOR_pS2448 | 0 | 0 | — | — |
| NADK_pS64 | 20 | 6 | 49 (3 Clones) | 1 |
| GSK3B_pS9 | 21 | 0 | | N.T. |
| EIF2S1_pS51 | 2 | 48 | 16 (1 clone) | 34 |

TABLE 3
A Phospho-Focused Antibody Library Results in
Enhanced Biopanning - Phospho-Tyrosine Targets

| | Total | Characterization | |
|---|---|---|---|
| | Number of | | Titration |
| DPAC1 Target | pTyr Hits | Sequencing | ELISA |
| hAbl1 pY245 | 12 | 2 | 1 |
| Integrin B1 pY783 | 0 | 1 (PYP, | P |
| | | preferred) | independent |
| hCortactin pY421 | 73 | 3 | 1 |
| Abl2 pY272 | 19 | 2 | 1 |
| hCortactin pY466 | 3 | 2 | 1 |
| p190RhoGAPApY1105 | 5 | 3 | 2 |
| Abl pY412/Y439 | 0 | N.T. | N.T. |

Additional methods of producing recombinant antibody libraries can be used. For example, in vivo methods for the production of recombinant antibodies can also be used (FIG. 10 and FIG. 14). Data from animal-derivedd phospho-specific antibodies indicate about a 66% success rate against 6 test RAbMab clones in finding phospho-independent or serine-specific antibodies.

A highly diverse and compact Ab library similar to nature can be successfully constructed from a large number of mutations in a single Ab framework. It has been estimated that the potential human Ab repertoire is at least 10¹¹; however, the number of Ab specificities present at any one time is limited by the total number of B cells in an individual, as well as by each individual's encounters with antigens, which is approximately 10⁸ different specificities at any one time. This is one hundredth the diversity of the library described in one embodiment herein. Therefore, by performing one-round of bio-panning with this library, one can better mimic natural selection and obtain a diverse pattern of binding Abs. Even if the size of the library is essential for successful isolation of high affinity Abs, high affinity is not necessary the pre-requisite for successful application of an Ab. This is because other properties such as specificity, expression level and stability are also important.

The affinity of the Ab can be further enhanced by a modified affinity maturation approach (FIG. 5). This method involves the introduction of diversity into the Ab genes by error-prone PCR, with affinity selection of the variants with decreasing amounts of antigens. The affinity maturation process is useful either for improving the affinity of Ab that is selected from naive phage library of medium size that has lower binding affinity, or for generating “super” Abs to be used in certain applications, such as diagnostic or immunotherapy. It has been reported that phage Abs with approximately tenfold higher affinities (10⁻¹¹ M) than the ceiling affinity that can be obtained from in vivo selection of B-cell (10⁻¹⁰ M) have been made by in vitro affinity maturation.

### Example 3

### Screening of the Libraries

As a positive control for the screening experiments, anti-pSer Ab, AXM1293, which has an affinity for a pSer-containing peptide of approximately 1×10⁻⁷ M will be used. Preliminary alanine scanning data has supported the computational model that H2 residues bind to the phosphorylated residue and interact with the main chain of the peptide (FIG. 2). The data demonstrate (FIG. 5) that even a library of size 10⁸ is enriched for phospho-binders over a larger 10¹⁰ non-focused library.

### Phage Display Screen

To select for scFvs that recognize pSer or pTyr with specific-sequence context surrounding the post-translational modification, the libraries will be screened against a collection of pSer and pTyr peptides. For example, Akt1 pSer473, Rb pSer780, and CREB pSer133 will be used for the pSer library screen, and Her2 pTyr1222, Myb pTyr11, and Esr1 pTyr537 will be used for the pTyr library screen. Additional peptides can also be screened as further controls.

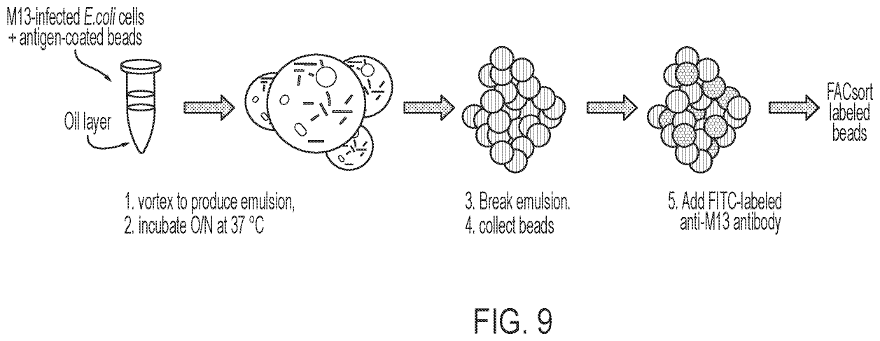

The same peptides lacking the phosphate groups will be added in solution as a competitor to select for scFvs that are selective for the phosphorylated peptide. The libraries will be screened by both standard phage display (FIG. 8) and using a micro-emulsion screening approach (FIG. 9). Using the emulsion approach, a more diverse set of binders can be recovered because clones are individually interrogated against the antigen within separate compartments in a water-in-oil emulsion.

Additional libraries will be created and screened wherein the electrostatic charges near the context-specific groove are changed.

### Preliminary Analysis of Discovered Antibodies and Antibody Characterization

Analysis of the data shows that often Abs directed against peptides are peptide-specific because the antibodies bind to the carboxylate at the end of the peptide. Where the carboxylate is usually distal in the full-length (FL) protein. Peptide-only anti-phospho-amino acid binders can be avoided by screening the second round against a peptide that is 5-6 amino acids longer than the initial binder.

Typically, 440 or more individual clones isolated from the screen will be analyzed by phage ELISA against the pSer or pTyr-containing peptides, their non-phosphorylated counterparts and control peptides and proteins. Clones with an ELISA signal >5-fold over background and at least 20% inhibition in the competition ELISA with its non-phosphorylated peptide derivative will be expressed in E. coli and purified by, methods in the art, such as, for example metal affinity chromatography.

The soluble scFvs and IgGs will be validated by various methods. For example, the phosphorylation state of the proteins in the lysates will be confirmed using, when available, existing protein-specific anti-pSer or anti-pTyr Abs commercially available from either Abcam or Cell Signaling. The validated scFvs will be converted to IgGs and expressed by transfection in CHO cells (FIG. 6).

### Example 4

### Generation of Modification-Specific Clonotypes (MSCs)

This example describes, among other things, methods to generate modification-specific clonotypes (MSCs). MSCs, as used herein, are antibodies that differ in a few amino acids and bind differentially to the post-translational modification state of the same epitope. In this example, the MSCs described are those that bind differentially to phosphorylated states of the same epitope.

To find pan-phosphate-binding Abs (i.e., binding to both states of phosphorylation at the phosphosite) site-directed mutagenesis will be used to place non-charged amino acids (either alanine- or glycine- into the binding pocket) at structurally-predicted and experimentally-determined sites in the Ab phosphosite binding pocket that are determined to interact with the phosphate. To find non-phosphate-binding (i.e., binding to only non-modified serine or tyrosine at the phosphosite) site-directed mutagenesis will be used to place negatively-charged amino acids (either aspartic- or glutamic-acid, which presumably would repel a phosphate at the binding pocket) at structurally-predicted and experimentally-determined sites in the phosphosite binding pocket. Directed evolution of these mutated Abs will be used and these mutated Abs will be screened against peptides wherein the phosphorylated amino acid in the peptide is now unmodified.

### Phage Display Screen, Antibody Characterization and Validation

Similar methodology will be used as described in the above-Phage Display Screen section except that the phosphate-binding amino acids in the Ab will be specifically changed (i.e., to either ala, gly, asp or glu). If there are no hits identified under the experimental conditions, we will vary the screening stringency.

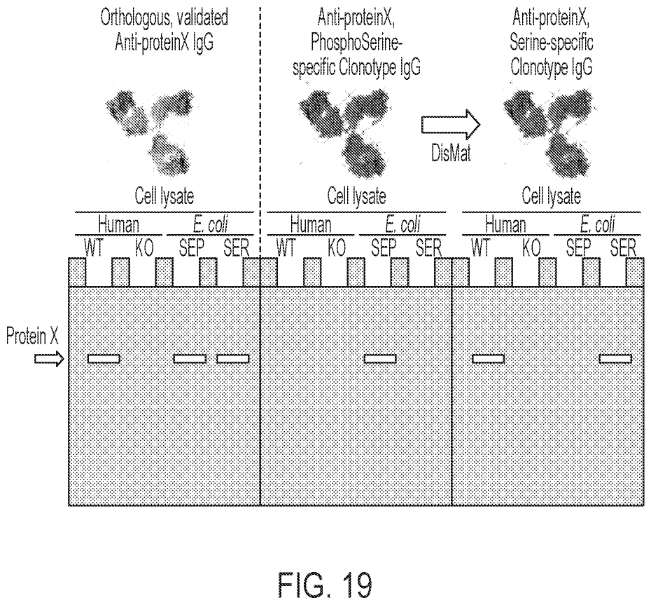

For antibody characterization, individual clones isolated from the screen will be analyzed by phage ELISA against either the pSer or pTyr-containing peptides and their non-phosphorylated counterparts. For antibody validation, the soluble MSC scFvs will be initially validated by Western blot against cell lysates over-expressing the targeted proteins without (and with) serine or tyrosine phosphorylation (see, e.g. FIGS. 13 and 19). The phosphorylation state of the proteins in the lysates will be confirmed using existing protein-specific anti-pSer or anti-pTyr Abs commercially available from Abcam and Cell Signaling. For example, FIG. 19 shows that where a knock-out cell line is available, four different cell lysates cab be produced: 1) wild-type (wt) human cell lysate, (2) knock-out (KO) cell line lysate, (3) protein X expressed in a SEP-suppressing E. coli (SEP) and (4) a wt E. coli strain (SER). If the orthologous, phosphoserine-MSC and serine-specific MSC antibodies give the pattern seen in the FIG. 19, then that supports the finding that the MSC antibodies are validated. In FIG. 19, it is assumed that protein X is not phosphorylated in wildtype. The validated scFvs will be converted to IgGs by cloning the variable heavy and light chain genes into our pMINERVA vectors and expressing them by transient transfection of CHO cells (FIG. 6).

### Example 5

### Directed Phospho-Ab Clonotypes (DPAC)

Antibody phosphate binding sites can be designed as a localized module and used as an efficient means of deriving phospho-specific antibodies. The strategy presented herein is to produce a phospho-focused enriched phage display antibody library where the CDR-H2 of the antibody is held constant for phospho-preferred and phospho-specific binding activity. This library is used in phage biopanning to direct the antibodies displayed on the M13 gpIII protein to synthetic peptides containing a pre-specified phosphorylated amino acid.

### Results of Preliminary DPAC Studies

A set of 20 pairs (i.e., a pair consists of both a Sep-containing and a native sequence) of peptides were synthesized. The 20 Sep-peptides were screened using AXM40 and AXM41 libraries. The results are shown in Table 4 below.

TABLE 4
Results from Screening of Phage Libraries

| | Competition | | | no competition | | |
|---|---|---|---|---|---|---|
| AXL40 | hits | P-specific | P-prefer | hits | P-specific | P-prefer |
| P4_p_Smad2 | 43 | 35 | 3 | 45 | 24 | 5 |
| P4_np_Smad2 | 3 | | | 21 | | |
| P5_p_CREB | 81 | 2 | 18 | 74 | 0 | 7 |
| P5_np_CREB | 78 | | | 82 | | |
| P6_p_HtrA2 | 5 | 5 | 0 | 35 | 0 | 3 |
| P6_np_HtrA2 | 3 | | | 35 | | |
| P15_p_p70s6_444 | 3 | 8 | 0 | 47 | 11 | 2 |
| P15_np_p70s6_444 | 0 | | | 40 | | |
| P11_p_akt1_473 | 25 | 15 | 4 | 50 | 4 | 26 |
| P11_np_akt1_473 | 11 | | | 43 | | |
| P3_p_RIPK3 | 35 | 3 | 5 | 72 | 1 | NA |
| P3_np_RIPK3_NB | 31 | | | 73 | | |
| P10_p_Sgk1 | 23 | 8 | 1 | 50 | 21 | NA |
| P10_np_Sgk1_NB | 14 | | | 21 | | |
| P2_p_RIPK3 | 70 | 68 | 4 | 76 | 70 | NA |
| P2_np_RIPK3_NB | 3 | | | 16 | | |
| P14_p_p53_15 | 76 | 7 | 7 | 86 | 1 | NA |
| P14_np_p53_15_NB | 69 | | | 86 | | |
| P13_p_p70s6_389aka412 | 6 | 6 | 0 | NA | NA | NA |
| P13_np_p70s6_389aka412_NB | 0 | | | NA | NA | |

‘Hits’ from the discovery screen were subsequently tested against the non-phosphorylated native sequences. Three classes of hits were observed: (i) phospho-independent, where the Ab bound equally to both the phospho- and non-phosphorylated peptide, (ii) phospho-specific, which bound the phospho-peptide >10-fold better than to the native peptide, and (iii) phospho-preferred, which bound to both the phosphorylated and the non-phosphorylated peptide, with >5-fold preference for the phosphorylated peptide. The phospho-preferred candidates were first pursued because they already: (1) bind to the native sequence, and (2) because they are affected by the phosphate, are presumed to be most likely localized near the Sep. The phospho-preferred are now being converted into IgGs, purified and validated against full length protein. If needed, these discovery hits can be affinity matured by AXM mutagenesis to derive antibodies with increased binding affinity to the native sequence. The scFv or Fabs converted to IgGs will be further validated.

### Summary of Preliminary DPAC Studies.

Phospho-focused libraries with an inherent propensity to bind phosphorylated peptides can be efficiently produced with a natural phosphate-binding motif in CDR H2. The amino acids in CDR H2 of an anti-Myb Sep (Sep11)-specific scFv (AXM1293) are critical for binding the phosphorylated Myb₁₋₂₀ peptide (FIG. 13). Several pilot libraries have been manufactured where H2 was held constant and residues on L3, H3 and L2 were randomized. These pilot libraries have already displayed a promising success rate (10 out of 10 tested) against 20-25mer phosphorylated-peptides compared with our traditional library (generally yielding a <50% success rate against similarly-sized peptides, see Table 4).

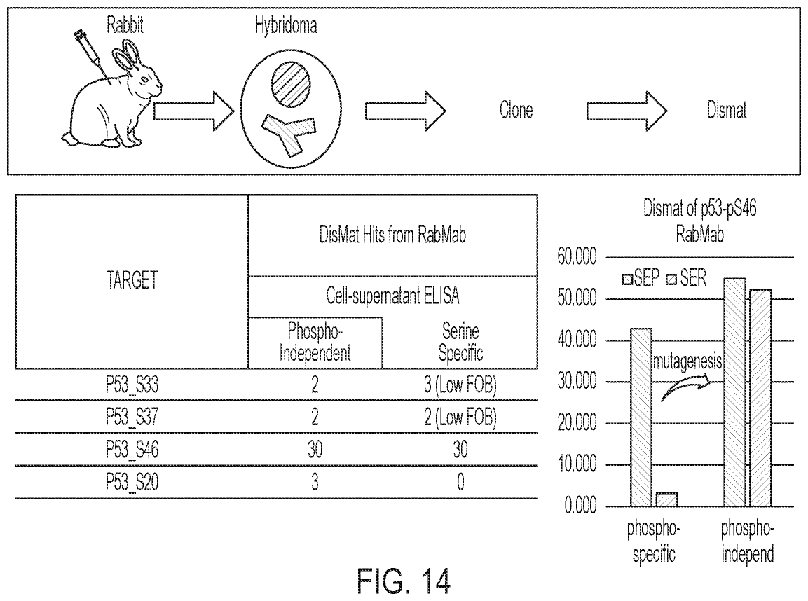

FIG. 15, panels A and B show another example of DPAC generated anti-akt473 antibody. FIG. 15 (panels A and B) shows the discovery and discovery maturation (“DisMat”) of anti-akt473. For this study, a phosphopeptide corresponding to the akt473 site was screened against a phospho-focused library and a phospho-specific clone was isolated. This clone was then mutagenized and re-screened against an unmodified peptide.

### Example 5

### Design of Anti-Enzyme Antibody Steric Inhibitors

This example describes the production of steric enzyme inhibitors. In some embodiments, the steric enzyme inhibitors can be used in cell- and enzyme-based screening assays.

It is often difficult to generate recombinant antibodies to (or at the least adjacent to-) a specific amino acid on a protein or peptide. Described herein and in the above examples are methods that allow for the generation of recombinant antibodies to a specific amino acid on a protein or a peptide. As described in the examples above, using function-based structure models and alanine scanning, a general mode for phospho-amino acid- and context sequence-binding by IgG and scFv Abs has been identified.

Antibodies directed against a phospho-site on a protein or peptide often derive from a particular CDR-H2 germline that predisposes these Abs to bind to phosphorylated amino acids. These Abs bind via two regions on the Ab: (1) a specific CDR-H2 sequence that can be considered the “phospho-specific” binding site region, and (2) a second region, composed of one or more of the remaining five CDRs, binds to the ‘context’ sequence. The context sequence causes the resulting Ab to be specific for the phospho-protein or -peptide antigen. Ab libraries can be made whereby the CDR-H2 responsible for specific phospho-binding activity is held to a constant amino acid sequence. With at least three specific amino acids in CDR-H2 responsible for interaction with the phosphate, these libraries are nearly ten-thousand-fold (20³) enriched for anti-phospho amino acid binding over ordinary screening libraries.

One method herein is to produce a phospho-focused enriched library where the CDR-H2 is held constant for phospho-specific binding activity. This library will be used in phage biopanning to direct the Abs displayed on the M13 gpIII protein to peptides containing a pre-specified phosphorylated amino acid. In a complementary approach, protein structure analysis will be used to define one or more surface-exposed amino acid loops adjacent to- or (conceivably) within an enzyme active site. Synthetic peptides composed of these loop sequences will be synthesized in pairs composed of: (1) the native sequence, and (2) a peptide where the central amino acid of the native sequence is replaced with either a phosphoserine or phosphotyrosine. In one embodiment, loop selections will be biased to loops where the central amino acid actually is either a serine or tyrosine. It has been noted that these amino acids are often found on protein surfaces. The anti-phospho-enriched library will first be biopanned against the phospho-containing peptide to derive initial discovery clones. Then, using directed evolution, the anti-phospho Ab will be evolved against the native sequence. Finally, these evolved Abs that are able to bind to the native unphosphorylated context sequence will be tested for their ability to inhibit enzyme activity and, if needed, affinity matured for higher affinity.

In some embodiments, this method further includes choosing loops where the central amino acid is replaced with either a phosphoserine or phosphotyrosine, regardless of what amino acid is present in the native sequence. This will allow for screening several peptides for any given protein sequence if any one amino acid using this method fails to yield an appropriate binder. By moving the phosphorylated amino acid up- or down-stream of a given position within the peptide will allow for increased opportunities for developing the loop-specific Abs.

Described herein are methods of making anti-enzyme antibody steric inhibitors. To this end, phospho-focused specific libraries based on an Ab framework with a pre-existing propensity to bind phosphorylated amino acids and a general mode to bind context sequences will be used. This phospho-specific library will be used to direct Ab-binding to phospho-modified peptides composed of the amino acid sequence of surface-exposed loops surrounding the active sites of enzymes. This will allow for the rapid generation of reversible inhibitors with utility in biochemistry and pharmacology.

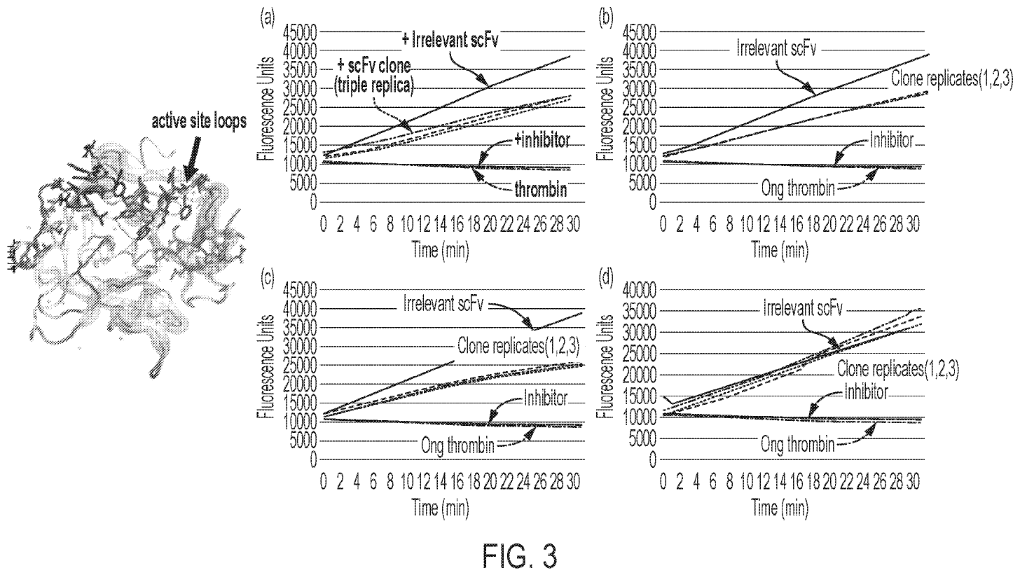

Steric inhibitor antibodies were successfully produced against human thrombin protein (FIG. 3). To this end, peptides corresponding to overlapping portions of a selected thrombin sequence surrounding the thrombin active site were synthesized. The synthesized peptides were engineered to incorporate a phosphorserine (GGGSGGSWGEGCDR(pSer)GKYG). The scFv hits were then matured against the native sequence (underlined): qmgivswgegcdrdgkygfythvfr. Of four scFv binders shown, three (FIG. 3, panels a, b, and c) inhibited thrombin activity, whereas one of the chosen peptide-binders, (FIG. 3, panel d), did not.

### Design of the Library, Library Construction, Design of the Phosphorylated Immunogen, and Library Screen

pSer, pTyr and pSer/pTyr libraries will be designed and constructed as described in the examples above. The pSer, pTyr and pSer/pTyr libraries will then be contacted with a phosphorylated immunogen.

In some embodiments, the enzyme targets will focus on those possessing the following desirable properties: (i) commercially available or relatively easy-to-produce purified protein, (ii) relatively simple-to-perform enzymatic assays (for example gel-based, fluorescence-based or colorimetric assays), (iii) structural information available in a public database, (iv) benchmark inhibitors (e.g. steric) known and available, and (v) surface exposed loops near-adjacent to the active site that contain either a serine or tyrosine. In some embodiments, classes of the proteins that have all or almost all of these properties include: polymerases, restriction enzymes, phosphatases, kinases, ATPases, Luciferases, and the like. The structures of the enzymes will be used to identify and choose surface loops.

As in the examples above, the affinity of the antibodies can be enhanced by affinity maturation (FIG. 7). For the screening process, the libraries will be screened using both standard phage display (FIG. 8) and a micro-emulsion screening approach (FIG. 7).

Candidate scFvs can be converted to IgGs as needed (FIG. 6). This conversion of candidate scFvs to IgGs allows for a 4- to 10-fold increase in binding affinity due to an “avidity effect” that occurs as a result of the dimerization of the scFv variable regions in an IgG.

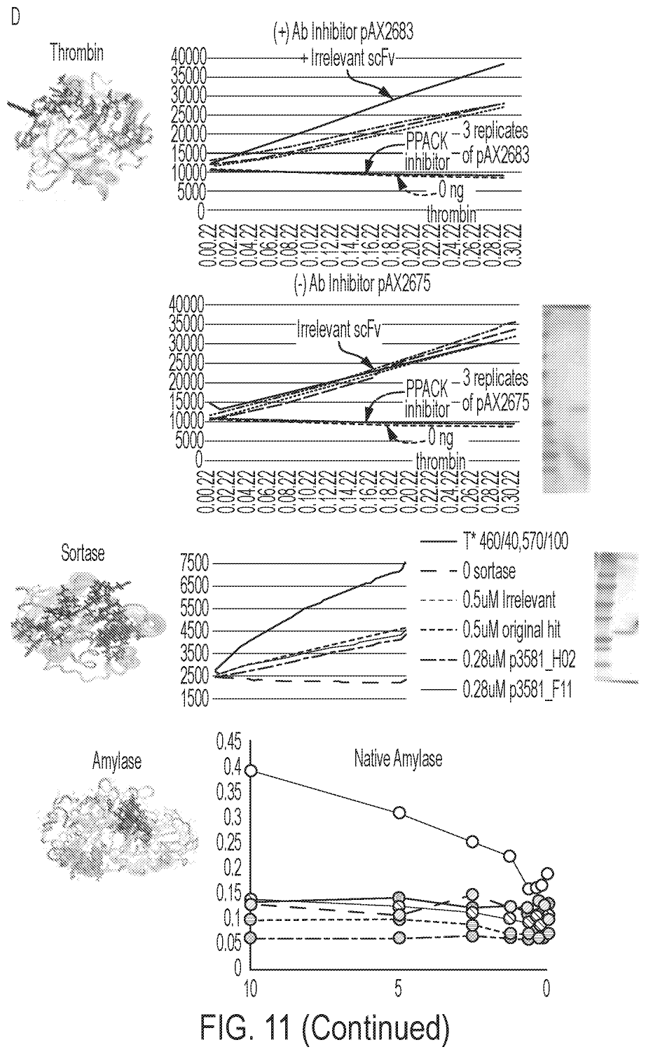

Exemplary antibodies generated using phage-derived IgG antibodies as described herein are presented in FIG. 12. For example, as shown in FIG. 12, antibodies derived from phage-derived IgG antibodies in accordance with the methodology described herein have been validated in immunohistochemistry applications (FIG. 12, panel A), Western Blot applications (FIG. 12, panel B, flow cytometry applications (FIG. 12, panel C), and in chromatin immunoprecipitation assays (ChiP) (FIG. 12, panel C).

FIG. 12, panel A shows a representative immunohistochemistry application of a constructed antibody against Myelin Basic Protein in SK-N-SH (human neuroblastoma cell line) cells. The cells were fixed with 4% formaldehyde (10 min), permeabilized with 0.1% Triton X-100 for 5 minutes and then blocked with 1% BSA/10% normal donkey serum/0.3 glycine in 0.1% PBS-Tween for 1 hr. The cells were then incubated overnight at +4° C. with ab209328 at a 5 μg/ml concentration, then detected with a donkey anti-human (Alexa Fluor 488) secondary Ab at a 1/2000 dilution (shown in green). Nuclear DNA was labeled with DAR (shown in blue) and ab195889, mouse monoclonal to alpha Tubulin (Alexa Fluor 594), at a 1/250 dilution (shown in red).

FIG. 12, panel B shows a representative Western Blot using an anti-histone H2B antibody. All lanes: Anti-Histone H2B Ab [IGX4228R-1] (ab213225) at 0.25 μg/ml. Lane 1: CTH (Calf Thymus Histone) at 0.5 μg. Lane 2: HeLa (Human epithelial carcinoma cell line) Whole Cell Lysate at 20 μg. Lane 3: HeLa (Human epithelial carcinoma cell line) Nuclear Lysate at 10 μg. Lane 4: NIH 3T3 (Mouse embryonic fibroblast cell line) Whole Cell Lysate at 20 μg. Lane 5: NIH 3T3 (Mouse embryonic fibroblast cell line) Nuclear Lysate at 10 μg. Lane 6: Histone H2B Recombinant Protein at 0.1 μg Lane 7: Histone H3 Recombinant Protein at 0.1 μg. Secondary Peroxidase AffiniPure Goat Anti-Rabbit IgG (H+L) at 1/50000 dilution. Developed using the ECL technique. Performed under reducing conditions. Predicted band size: 14 kDa Observed band size: 17 kDa. Exposure time: 1 minute. This blot was produced using a 4-12% Bis-tris gel under the MES buffer system. The gel was run at 200V for 35 minutes before being transferred onto a Nitrocellulose membrane at 30V for 70 minutes. The membrane was then blocked for an hour using 3% milk before being incubated with ab213225 overnight at 4° C. Ab binding was visualized using ECL development solution ab133406.

FIG. 12, panel C shows a representative flow cytometry plot using an anti NTSR antibody. Briefly, a cell line overexpressing the GPCR NTSR1 (black) and a negative control null cell line (in blue, upper, and purple, bottom) were mixed with either fluoresceinated NTSR1 ligand (top) or scFv E1 (bottom) and subjected to flow cytometric analysis.

FIG. 12, panel D shows representative chromatin immunoprecipitation results (ChIP) using a phage-derived IgG antibody. Brifely, Chromatin was prepared from HeLa cells according to the Abcam X-ChIP protocol. Cells were fixed with formaldehyde for 10 minutes. The ChIP was performed with 25 μg of chromatin, 2 μg of ab213288 (blue), and 20 μl of Protein A/G sepharose beads. No Ab was added to the beads control (yellow). The immunoprecipitated DNA was quantified by real time PCR (Taqman approach). Primers and probes were located in the first kb of the transcribed region.

### Example 7

### Design of Anti-Idiotype and Enzyme Inhibitor Antibodies

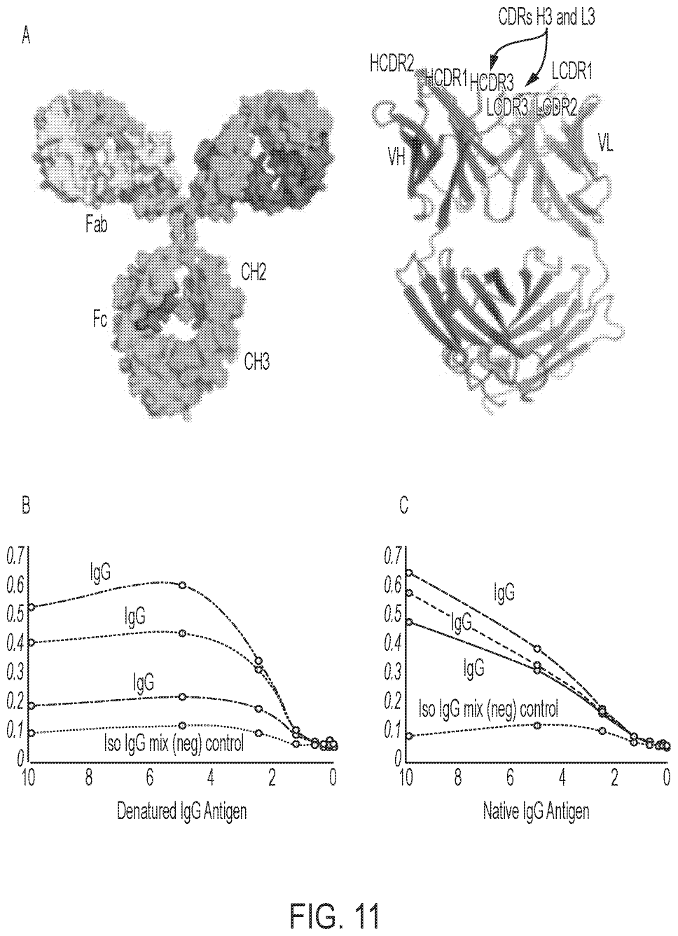

This example describes and demonstrates the generation of anti-idiotype antibodies. This example also describes and demonstrates the generation of enzyme inhibitor antibodies that inhibit sortase, thrombin and amylase (FIG. 11, panel D). As a proof of concept, these enzyme were chosen, however, as will be appreciated by those of ordinary skill in the art, the methodology presented herein can also be applied to targeting other enzymes or other antibodies (e.g. anti-idiotype antibodies) that are known in the art.

Anti-idiotype antibodies were constructed against a selected antibody (FIG. 11, panels A-C). Both the anti-idiotype antibodies and the enzyme inhibitor antibodies were constructed using the same workflow as shown in the flow diagram depicted in FIG. 2. Briefly, for the generation of anti-idiotype antibodies, a base sequence from CDRs H3 and L3 was selected to create separate, modified peptides. The modified peptides were synthesized to include phosphoserine within its sequence. A phage library was screened against the modified peptide, resulting in the isolation of antibodies that were specific to the CDRH3 of the selected antibody, thus generating anti-idiotype antibodies. Binding assays show binding of the anti-idiotype antibodies (FIG. 11, panels A-C).

Using the methodology described herein, and depicted in FIG. 2, antibodies were also generated against the following enzyme targets: sortase, thrombin and amylase. Binding assays were obtained from the isolated antibodies and confirm binding and enzyme inhibition upon contact with the isolated antibodies that were generated (i.e. sortase, thrombin, and amylase) (FIG. 11D).

### Example 8

### Delayed Emulsion Infectivity (DEI): Recombinational Biopanning Strategy

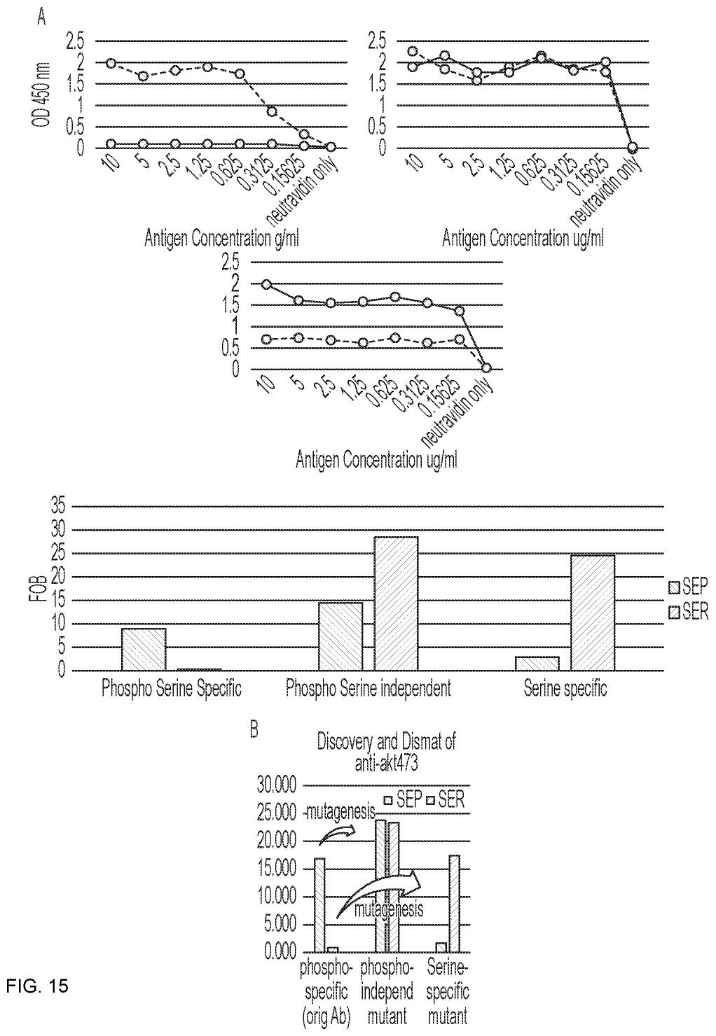

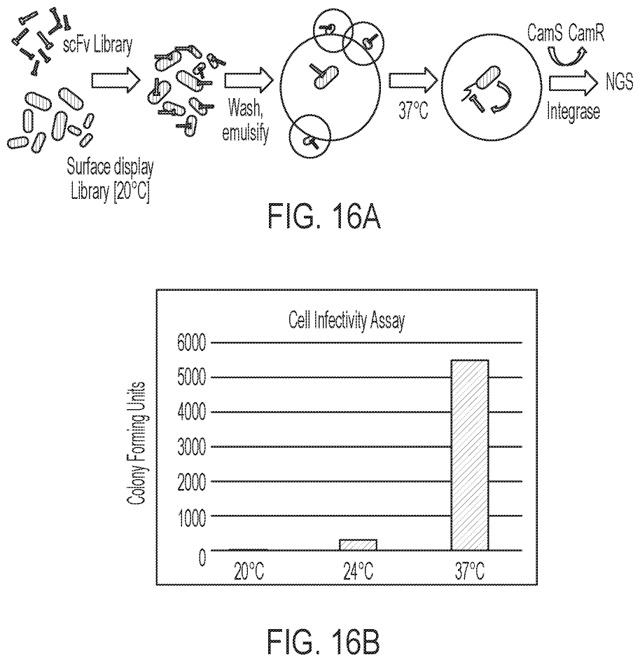

FIG. 16, panel A shows a schematic illustrating the Delayed Emulsion Infectivity Recombinational biopanning strategy. Briefly, the DEI biopanning strategy is summarized as follows. An E.coli surface-display Lpp'ompA-fusion library of ca. 104 different Ags is grown at 16° C. to suppress F-pilus (the M13 receptor on the cell surface) expression and clonal expansion. A phage-display Ab-library of >1010 complexity is combined with the bacterial library in bulk solution for a period sufficient for Ag-Ab interaction to occur, then washed to remove any unattached phage. Trypsin (to specifically cleave the Ab from the gpIII protein for added specificity and increased detachment efficiency) is added and the cells (and any attached phage) are quickly compartmentalized by vortexing the mix to encapsulate the cells within individual droplets of a water-in-oil emulsion. The emulsion is shifted to 37° C. to induce expression of the F-pilus and detachment of the phage particles. Detached phage infect the bacteria via the induced F-pilus. The emulsion is broken and the cells grown on LB+Cam plates to prevent clonal expansion. Plasmid DNA from the pools is isolated. The region of DNA between the 5′ side of the scFv gene and the 3′ side of the epitope is amplified using PCR. The PCR product is then analyzed by Next Generation sequencing. After contig assembly, specific clones are identified using the barcodes encoded into both the Ab and epitope constant regions. Antibodies against a specific epitope can be amplified using an epitope-specific primer. Specific antibodies can be amplified using unique pairs of barcode-complementing primers. Specific antibodies can also be sub-cloned into a suitable expression system. One barcode is placed at the 5′ end of the scFv gene and a second is placed 3′ of the epitope gene.

### Summary of DEI Studies

DEI was modeled using a set of 18 peptides. Next generation sequencing (“NGS”) and a barcoding scheme was used to process several million HiSeq sequencing reads. In generating phospho-focused libraries in E. coli, phages that bind non-specifically to the E. coli cell surface were subtracted-out and greatly reduced. Bioinformatic analysis comprising comparing hits from a single target Ag to a combination of all of the remaining Ags to reduce the noise in the experiments was also performed. Using the same library for all of the Ags in a single, multiplex screen allowed for visualization in silico of the clones that bind to both target N and the two types of non-target N class sequences (non-specific and enriched in all-others).

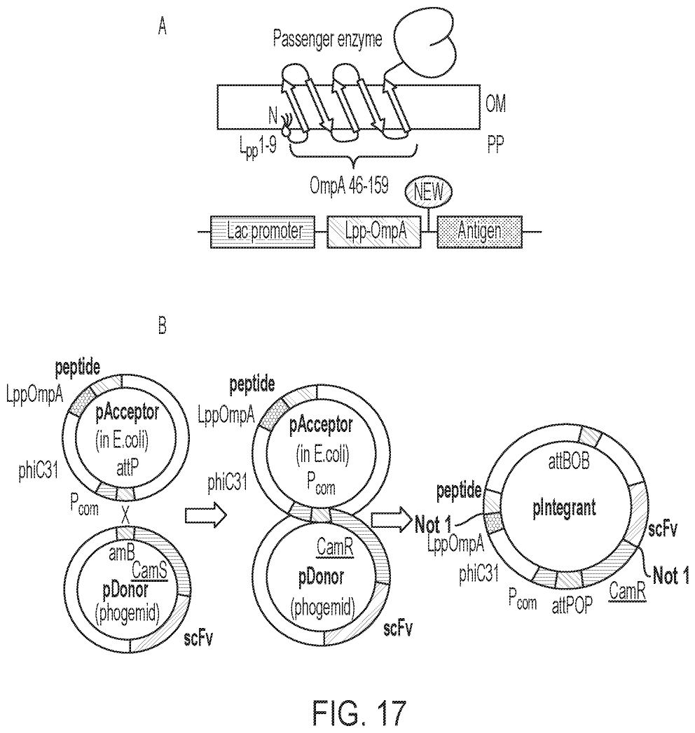

An embodiment of the bacterial display system associated with DEI is illustrated in FIG. 17. As shown in FIG. 17, one embodiment of the bacterial display system associated with DEI is the Lpp-OmpA bacterial display system.

Preliminary observations of the DEI system indicates that growth temperature has a direct effect on the infection ability by M13 (FIG. 16, panel B).

An embodiment of the bacterial display system associated with DEI is illustrated in FIG. 17.

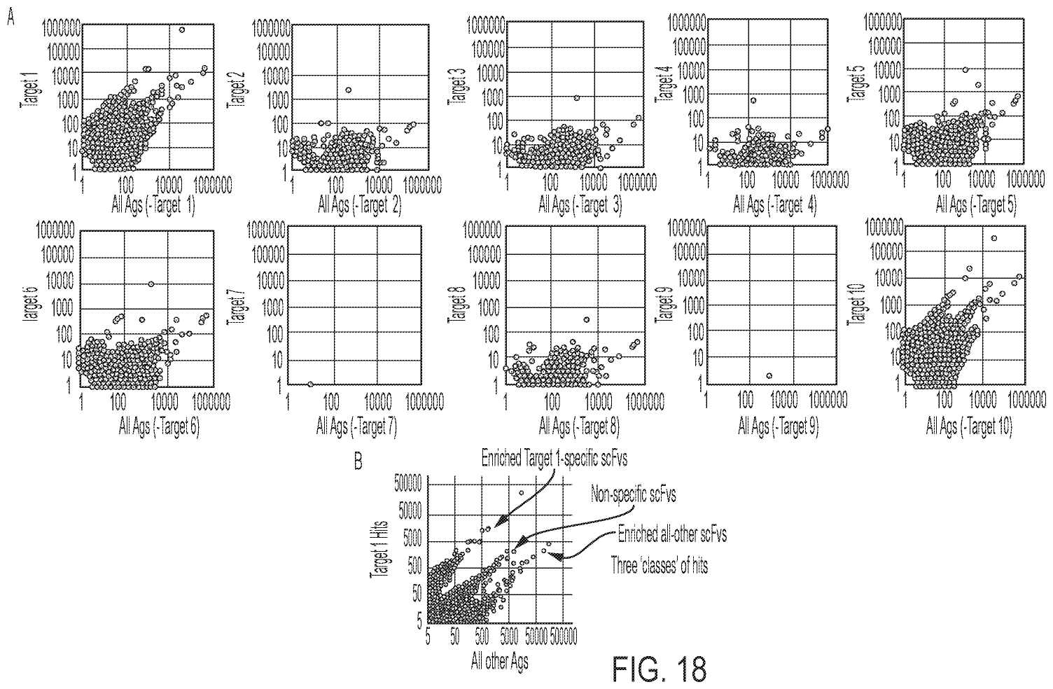

Results have been obtained from a multiplex of 18 separately displayed peptides on the surface of E coli. As shown in FIG. 18 (panels A and B), the results show over 1 million antibody next generation sequence reads for the first 10 peptides. The data obtained from this study showed three “classes” of groupings from this analysis: 1) enriched Target 1-specific scFvs; 2) non-specific scFvs; and 3) enriched all-other scFvs.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1-22. (canceled)

23. A method of generating a pair of modification-specific antibody clonotypes comprising:
(a) providing an antibody that specifically recognizes a PTM on a peptide or protein of interest;
identifying a PTM binding pocket of the antibody;
(b) introducing an amino acid that repels the PTM at one or more sites in the PTM binding pocket of the antibody that are determined to interact with the PTM;
(c) generating a library comprising candidate non-PTM binding antibodies by randomizing one or more regions outside the PTM binding pocket that bind to a context sequence adjacent to the PTM site;
(d) screening the library against the peptide or protein of interest without PTM, and
(e) selecting a non-PTM binding antibody to provide a pair of PTM-status-specific antibody clonotypes.

24. The method of claim 23, wherein the PTM site is a naturally occurring PTM site.

25-30. (canceled)

31. The method of claim 23, wherein the one or more sites within the PTM binding pocket are structurally-predicted.

32. The method of claim 23, wherein the one or more sites within the PTM binding pocket are experimentally-determined.

33. (canceled)

34. The method of claim 23, wherein the PTM is phosphorylation, glycosylation, or sialylation.

35-37. (canceled)

38. The method of claim 23, wherein the amino acid that repels the PTM is aspartic or glutamic acid.

39. (canceled)

40. The method of claim 23, wherein the PTM is retinylidene Schiff base formation or arginylation.

41. (canceled)

42. The method of claim 23, wherein the positively charged amino acid that repels the PTM is lysine, arginine, or histidine.

43-44. (canceled)

45. The method of claim 23, wherein the amino acid that repels the PTM is introduced by a suppressor tRNA or site-directed mutagenesis.

46. The method of claim 23, wherein the PTM is hydrophobic and the amino acid that repels the PTM is arginine, asparagine, aspartate, glutamine, glutamate, histidine, lysine, serine, or threonine.

47-48. (canceled)

49. The method of claim 23, wherein the PTM is hydrophilic and the amino acid that repels the PTM is glycine, alanine, valine, leucine, isoleucine, proline, phenylalanine, methionine or tryptophan.

50-53. (canceled)

54. The method of claim 23, wherein the context sequence comprises 3-15 residues upstream or downstream to the PTM site.

55. The method of claim 23, wherein the one or more regions outside the PTM binding pocket that bind to the context sequence are randomized by error-prone rolling circle amplification (RCA).

56. The method of claim 23, wherein the one or more regions outside the PTM binding pocket that bind to the context sequence are randomized without altering the PTM binding pocket.

57. The method of claim 23, wherein the library of step (c) is a phage display library.

58. (canceled)

59. The method of claim 23, wherein the step of screening the library comprises emulsion based whole cell panning.

60. (canceled)

61. The method of claim 23, wherein the method further comprises a step of validating the selected pair of PTM-status-specific antibody clonotypes, wherein the validating step is preferably high throughput.

62. (canceled)

63. The method of claim 61, wherein the PTM is phosphorylation and the high throughput validating step involves the use of a cell line that incorporates phospho-serine or phospho-tyrosine into suppressible amber (UAG) stop codons, thereby producing phosphorylated proteins for validating pan-PTM binding antibodies or non-PTM binding antibodies.

64. The method of claim 63, wherein the cell line is E. coli or an insect cell line.

65-162. (canceled)
